(19) United States
(12) Patent Application Publication
(10) Pub. No.: US 2025/0205191 A1
(43) Pub. Date: Jun. 26, 2025

(54) R(-)-MDMA COMPOSITIONS AND METHODS OF TREATMENT

(71) Applicant: EmpathBio, Inc., New York, NY (US)

(72) Inventors: Srinivas G. RAO, Encinitas, CA (US); Glenn SHORT, Encinitas, CA (US)

(21) Appl. No.: 18/990,840

(22) Filed: Dec. 20, 2024

Related U.S. Application Data
(60) Provisional application No. 63/682,179, filed on Aug. 12, 2024.
(60) Provisional application No. 63/614,046, filed on Dec. 22, 2023.

(51) Int. Cl. A61K 31/36; A61P 25/22

(52) U.S. Cl. CPC A61K 31/36; A61P 25/22

(57) ABSTRACT

The present disclosure relates to methods of treating an anxiety disorder, including social anxiety disorder, comprising administering a composition comprising an enantiomerically enriched form of R(−)-MDMA.

### CROSS-REFERENCE TO RELATED APPLICATIONS

This application claims the benefit of and priority to U.S. Provisional Application No. 63/614,046, filed Dec. 22, 2023; and U.S. Provisional Application No. 63/682,179, filed Aug. 12, 2024, the contents of each of which are hereby incorporated by reference in their entirety for all purposes.

### BACKGROUND

3,4-methylenedioxymethamphetamine (MDMA) is a psychoactive compound that affects mood, perception, and increases prosocial feelings and behaviors. It is a ring-substituted phenethylamine possessing a complex pharmacological profile that is dominated by its effects as a monoamine releaser and reuptake inhibitor. Its prominent serotonergic effects differentiate it from amphetamine and methamphetamine, which primarily act via dopaminergic and norepinephrine mechanisms of action. MDMA is considered the prototype for compounds referred to as entactogens, which means “to touch within” due to the induction of feelings of empathy and sociability. MDMA produces subjective effects that are unlike any of the classical psychostimulants or hallucinogens and is one of the few compounds capable of reliably producing prosocial behavioral states.

Racemic MDMA possesses a complex pharmacological profile as a result of enhanced triple monoamine (serotonin, norepinephrine, and dopamine) release coupled with inhibition of reuptake, blockade of vesicular storage of monoamines, inhibition of neurotransmitter oxidation by MAO-A, and a reversal of 5-HT transport into the neuron. These effects result in a net increase of monoamines in the synaptic cleft and prolong the duration of monoaminergic neurotransmission. MDMA also affects hormone secretion, promoting the release of oxytocin and arginine vasopressin (AVP).

MDMA has two stereoisomers S(+)-MDMA and R(−)-MDMA. The enantiomers have been reported to have differing effects on its monoaminergic targets, and differing pharmacological activities. The differing pharmacological and toxicological profile of the enantiomers indicate that R(−)-MDMA can provide a differentiated and improved therapeutic profile from either racemic MDMA or the S(+)-MDMA enantiomer.

The present disclosure provides compositions and methods that are effective in treating mental health diseases, disorders, and conditions, and the associated symptoms of such afflictions by providing enantiomerically enriched compositions of MDMA, i.e., compositions comprising specific ratios of the R(−) and S(+) enantiomers (up to 100% R(−)-MDMA).

### SUMMARY

The present disclosure provides methods of treating an anxiety disorder in a subject in need thereof, comprising administering to the subject a composition comprising an enantiomerically enriched form of R(−)-3,4-methylenedioxymethamphetamine (R(−)-MDMA), or a pharmaceutically acceptable salt or prodrug thereof, wherein the administration provides a R(−)-MDMA Tₘₐₓ of about 2 hours to about 10 hours and a R(−)-MDMA Cₘₐₓ of about 150 ng/ml to about 3000 ng/mL following administration. In embodiments, the anxiety disorder is social anxiety disorder. In embodiments, the subject has one or more condition comorbid with the anxiety disorder.

In embodiments, about 75 mg to about 350 mg R(−)-MDMA free base or an equivalent dose of the pharmaceutically acceptable salt or prodrug thereof is administered to the subject. In embodiments, about 225 mg R(−)-MDMA free base or an equivalent dose of the pharmaceutically acceptable salt or prodrug thereof is administered to the subject. In embodiments, the subject is administered 2 repeated doses of the composition at an interval of about 14 days to about 56 days. In embodiments, the subject is administered 2 repeated doses of the composition at an interval of about 28 days. In embodiments, the composition is orally administered.

In embodiments, the method of the present disclosure further comprises administering a second therapy. In embodiments, the second therapy comprises a psychological support.

In embodiments, the composition of the present disclosure comprises a weight ratio of R(−)-MDMA to S(+)-MDMA ranging from about 50.1:49.9 to about 100:0. In embodiments, the composition comprises R(−)-MDMA in an enantiomerically enriched form of ≥90%, ≥95%, ≥96%, ≥97%, ≥98%, ≥99%, ≥99.5%, ≥99.9%, or ≥99.99%, or 100% relative to total MDMA.

In embodiments, the subject has SAD that is not a performance only sub-type. In embodiments, the subject has a Liebowitz Social Anxiety Scale (LSAS) total score ≥70 prior to the administration. In embodiments, the administration provides a reduction in the subject's LSAS total score by at least about 20 compared to prior to the administration.

Other aspects and embodiments will be apparent to one skilled in the art in light of the detailed description and illustrative Examples that follow.

### BRIEF DESCRIPTION OF THE DRAWINGS

FIGS. 1A-1C show the number of neurites per neuron (FIG. 1A), total neurite length per neuron (FIG. 1B) and branchpoints per neuron (FIG. 1C) as percent change from the vehicle control (vehicle: 0.1% Sterile water vehicle control) in the presence of 8 concentrations of R(−)-MDMA and the positive control (+Cntl: 250 nM Donepezil) in rat embryonic cortical neuron cultures. Test article concentrations are expressed as log Molar concentration.

FIG. 2 shows a dose response assessment of head twitch (HTR) with 0-10 mg/kg R(−)-MDMA, IP, in male C57BL/6J mice.

FIGS. 3A-3B show freezing response (FIG. 3A) and locomotor activity (FIG. 3B) measured during extinction training (Day 1, left) and testing (Day 2, right) sessions in male C57Bl/6J mice receiving a single intraperitoneal administration of 10, 17 or 30 mg/kg R(−)-MDMA, 7.8 mg/kg racemic MDMA or 0.9% saline vehicle 30 min prior to extinction training on Day 1.

FIG. 4 illustrates plasma concentrations as a function of time for each cohort (placebo, 75 mg, 125 mg (fasted and fed), 175 mg, 225 mg R(−)-MDMA).

FIGS. 5A-5E shows the geometric means of plasma concentrations of R(−)-MDMA and its metabolite R(−)-MDA as a function of time for each cohort: 75 mg (FIG. 5A), 125 mg fasted (FIG. 5B), 125 mg fed (FIG. 5C), 175 mg (FIG. 5D), and 225 mg R(−)-MDMA (FIG. 5E).

FIG. 6 shows the SIRS results for each cohort (placebo, 75 mg, 125 mg (fasted and fed), 175 mg, and 225 mg R(−)-MDMA).

FIGS. 7A-7B shows show the changes from baseline in the concentration of prolactin (FIG. 7A), and oxytocin (FIG. 7B).

FIG. 8 shows 5-Dimensions-Altered States of Consciousness (5D-ASC) subscale ratings for subjects at various dosages and placebo.

FIG. 9 show 11-Dimensions-Altered States of Consciousness (11D-ASC) sub-scale ratings for subjects at various dosages and placebo.

FIG. 10 shows a plot of 11D-ASC sub-scale ratings for R(−)-MDMA at indicated dosages and placebo.

FIG. 11 shows a plot of 11D-ASC sub-scale ratings for R(−)-MDMA at 225 mg and 175 mg dosages, as well as psilocybin (at 20-30 mg), racemic MDMA (at 125 mg), and intravenous dimethyltryptamine (DMT-IV) (at 0.667 mg/min).

FIG. 12 shows Emotional Breakthrough Inventory (EBI) mean total score and mean item scores for each cohort (placebo, 75 mg, 125 mg (fasted and fed), 175 mg, and 225 mg R(−)-MDMA).

FIG. 13 shows the changes from baseline in MET LSmean scores for subjects at various dosages and placebo.

FIG. 14 shows the changes from baseline as measured by Self-Compassion Scale (SCS) for subjects at various dosages and placebo.

FIG. 15 shows changes from baseline 6.5 hours post dose in Challenging Experiences Questionnaire (CEQ) for subjects at various dosages and placebo.

FIG. 16 shows the study design for Phase 2 study.

### DETAILED DESCRIPTION

### I. Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 50.5.

The terms “administer,” “administering” or “administration” as used herein refer to administering a compound or pharmaceutically acceptable salt of the compound or a composition or formulation comprising the compound or pharmaceutically acceptable salt of the compound to a patient.

The term “subject,” “individual” and “patient” are used interchangeably herein, and refers to a human.

The terms “treating” as used herein with regard to a patient, refers to improving at least one symptom of the patient's disorder. Treating can be improving, or at least partially ameliorating a disorder or an associated symptom of a disorder.

The term “pharmaceutically acceptable” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form alkali metal salts of free acids and to form addition salts of free bases. Salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

The term “carrier” or “vehicle” as used interchangeably herein encompasses carriers, excipients, adjuvants, and diluents or a combination of any of the foregoing, meaning a material, composition or vehicle, such as a liquid or solid filler, diluent, excipient, solvent or encapsulating material involved in carrying or transporting a pharmaceutical agent from one organ, or portion of the body, to another organ or portion of the body. In addition to the adjuvants, excipients and diluents known to one skilled in the art, the carrier includes nanoparticles of organic and inorganic nature.

The term “effective amount” or “therapeutically effective amount” as used interchangeably in this disclosure and refer to an amount of a compound, or a salt thereof, (or pharmaceutical composition containing the compound or salt) that, when administered to a patient, is capable of performing the intended result.

### II. Compositions

In aspects, compositions of the disclosure comprise an amount of active ingredient (e.g., R(−)-MDMA or a pharmaceutically acceptable salt or prodrug thereof, in enantiomerically enriched forms). In embodiments, compositions of the disclosure comprise R(−)-MDMA in an enantiomeric excess relative to S(+)-MDMA, and a pharmaceutically acceptable carrier, wherein the amount of R(−)-MDMA relative to S(+)-MDMA is from about 50.01% to about 100% R(−)-MDMA. In embodiments, the amount of R(−)-MDMA relative to S(+)-MDMA is from about 65% to about 100% R(−)-MDMA.

In embodiments, compositions of the disclosure comprise R(−)-MDMA in an enantiomeric enrichment or purity form of ≥90%, ≥95%, ≥96%, ≥97%, ≥98%, ≥99%, ≥99.5%, ≥99.9%, ≥99.99%, or 100% (i.e., relative to total MDMA).

In embodiments, the composition comprises a weight ratio of R(−)-MDMA to S(+)-MDMA that is greater than 1. For example, the composition can comprise a weight ratio of R(−)-MDMA to S(+)-MDMA ranging from about 50.01:49.99 to about 100:0 (e.g., about 50.1:49.9, about 50.5:49.5, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, about 99:1, about 99.5:0.5, about 99.9:0.1, or about 100:0, among other ratios within these enumerated ratios).

In embodiments, compositions of the disclosure comprise a weight ratio of R(−)-MDMA to S(+)-MDMA ranging from about 90:10 to about 100:0 (e.g., a weight ratio of R(−)-MDMA to S(+)-MDMA of about 90:10, about 91:9, about 92:8, about 93:7, about 94:6, about 95:5, about 96:4, about 97:3, about 98:2, about 99:1, about 99.5:0.5, about 99.9:0.1, or about 100:0, amount other ratios within these enumerated ratios). In embodiments, the composition comprises a weight ratio of R(−)-MDMA to S(+)-MDMA ranging from about 99:1 to about 100:0. For example, the weight ratio of R(−)-MDMA to S(+)-MDMA comprises about 99.1:0.9, about 99.2 to 0.8, about 99.3 to 0.7, about 99.4 to 0.6, about 99.5 to 0.5, about 99.6, about to 0.4, about 99.7 to 0.3, about 99.8 to 0.2, about 99.9 to 0.1, or 100:0.

In embodiments, the composition comprises at least about 90% R(−)-MDMA and no more than about 10% S(+)-MDMA (e.g., at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, at least about 99.95%, at least about 99.96%, at least about 99.97%, at least about 99.98%, at least about 99.99% R(−)-MDMA, or 100% R(−)-MDMA; and less than about 10%, less than about 9%, less than about 8%, less than about 7%, less than about 6%, less than about 5%, less than about 4%, less than about 3%, less than about 2%, less than about 1%, less than about 0.1%, less than about 0.01% S(+)-MDMA, or 0% S(+)-MDMA).

### III. Dosage Regimens

The composition in accordance with the uses and methods described herein is effective for treating or alleviating one or more diseases, disorders, or conditions, or one or more symptoms associated therewith, in a subject as described herein. Specifically, the dosage of the composition to achieve a therapeutic effect will depend on factors such as the formulation and pharmacological potency of the composition.

In embodiments, the effective amount of the enantiomerically enriched R(−)-MDMA composition to be administered comprises a dose ranging from about 1 mg to about 1000 mg R(−)-MDMA free base or an equivalent dose of the pharmaceutically acceptable salt or prodrug thereof based on a typical (e.g., 60 kg or 70 kg) mammal subject, (e.g., a human subject). In embodiments, the therapeutically effective amount comprises a dose ranging from about 1 mg to about 750 mg, about 5 mg to about 500 mg, about 10 mg to about 400 mg, about 25 mg to about 300 mg, about 75 mg to about 225 mg, about 75 mg to about 850 mg, about 250 mg to about 850 mg, or about 250 mg to about 350 mg R(−)-MDMA free base or an equivalent dose of the pharmaceutically acceptable salt or prodrug thereof. In embodiments, the therapeutically effective amount comprises about 25 mg to about 350 mg R(−)-MDMA free base or an equivalent dose of the pharmaceutically acceptable salt or prodrug thereof. In embodiments, the therapeutically effective amount comprises a dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, or more R(−)-MDMA free base or an equivalent dose of the pharmaceutically acceptable salt or prodrug thereof.

In embodiments, the effective amount comprises a dose of about 0.01 mg/kg to about 20 mg/kg R(−)-MDMA free base or an equivalent dose of the pharmaceutically acceptable salt or prodrug thereof (e.g., about 1 mg/kg to about 15 mg/kg, about 5 mg/kg to about 15 mg/kg, about 10 mg/kg to about 20 mg/kg, about 3 mg/kg to about 15 mg/kg, or about 3 mg/kg to about 5 mg/kg MDMA free base or an equivalent dose of the pharmaceutically acceptable salt or prodrug thereof).

In embodiments, the effective amount comprises a dose of about 0.001 mg/kg to 100 mg/kg R(−)-MDMA free base or an equivalent dose of the pharmaceutically acceptable salt or prodrug thereof (e.g., about 0.001 mg/kg, about 0.01 mg/kg, about 0.1 mg/kg, about 0.25 mg/kg, about 0.5 mg/kg, about 0.75 mg/kg, about 1.0 mg/kg, about 1.5 mg/kg, about 2.0 mg/mg, about 2.5 mg/kg, about 3.0 mg/kg, about 3.5 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg MDMA free base or an equivalent dose of the pharmaceutically acceptable salt or prodrug thereof).

The pharmaceutical compositions of the disclosure can be formulated in a variety of unit dosage forms depending upon the method of administration. Suitable unit dosage forms, include, but are not limited to powders, tablets, pills, capsules, lozenges, sprays, granules, oral thin films for buccal and/or sublingual administration.

In embodiments, the effective amount is provided as a single dose or on regular schedule, i.e., on a daily, weekly, monthly, or yearly basis or an irregular schedule with varying administration days, weeks, months, etc. In embodiments, a subject is administered the composition of the present disclosure multiple times over a set period of time. In embodiments, the patient is administered the composition once every time period of between about 1 week and about 12 months for a set treatment period. In embodiments, the subject is administered the composition once every time period of between about 1 week and about 6 months for a set treatment period. In embodiments, the subject is administered the composition once every about 1 week, about 2 weeks, about 3 weeks, about 4 weeks, about 2 months, about 3 months, about 4 months, about 5 months, about 6 months, about 7 months, about 8 months, about 9 months, about 10 months, about 11 months, or about 12 months for a set treatment period. In embodiments, the subject is administered the composition once every about 2 weeks for a set treatment period. In embodiments, the subject is administered the composition once every about 4 weeks for a set treatment period. In embodiments, the subject is administered the composition once every about 6 weeks for a set treatment period. In embodiments, the subject is administered the composition once every about 2 months for a set treatment period. In embodiments, the subject is administered the composition once every about 3 months for a set treatment period. In embodiments, the subject is administered the composition once every about 6 months for a set treatment period. In embodiments, the subject is administered the composition once every about 14 days to about 56 days for a set treatment period. In embodiments, the subject is administered the composition once every about 14 days, about 21 days, about 28 days, about 35 days, about 42 days, about 49 days, or about 56 days for a set treatment period. In embodiments, the subject is administered the composition once every about 28 days for a set treatment period. In embodiments, the subject is administered from about 2 to about 20 repeated doses of the composition at an interval of about 14 days to about 56 days. In embodiments, the subject is administered 2 repeated doses of the composition at an interval of about 28 days.

To reduce the occurrence of possible side effect associated with the dose, an effective amount may be provided as a split dose, where the single dose is split into two doses, that are administered apart, usually over several hours. For example, a single dose may be split into two doses, administered 1 hour apart, 2 hours apart, 3 hours apart, 4 hours apart, 5 hours apart, 6 hours apart, 7 hours apart, 8 hours apart, or more. Alternatively, the therapeutically effective amount to be administered may vary. In aspects, the therapeutically effective amount for the first dose is higher than the therapeutically effective amount for one or more of the subsequent doses. In aspects, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every 2 weeks, about every 3 weeks, about every month, about every 2 months, about every 3 months and about every 6 months. The number and frequency of dosages corresponding to a completed course of therapy will be determined according to the judgment of a health-care practitioner.

In embodiments, the dosage regimens of the present disclosure provide an effective serum or plasma concentration of active agent in the subject. In embodiments, the administration of the compositions of the present disclosure provides a maximum plasma concentration Cₘₐₓ of R(−)-MDMA from about 150 to about 3000 ng/mL, about 200 to about 3000 ng/ml, about 250 to about 3000 ng/ml, about 300 to about 3000 ng/mL, about 350 to about 3000 ng/mL, about 400 to about 3000 ng/mL, about 450 to about 3000 ng/mL, about 500 to about 3000 ng/ml, about 550 to about 3000 ng/mL, about 600 to about 3000 ng/mL, about 650 to about 3000 ng/ml, about 700 to about 3000 ng/mL, about 750 to about 3000 ng/mL, about 800 to about 3000 ng/mL, about 850 to about 3000 ng/mL, about 900 to about 3000 ng/mL, about 1050 to about 3000 ng/ml, about 1100 to about 3000 ng/ml, about 1150 to about 3000 ng/mL, about 1200 to about 3000 ng/ml, about 1250 to about 3000 ng/mL, about 1300 to about 3000 ng/ml, about 1350 to about 3000 ng/ml, about 1400 to about 3000 ng/ml, about 1450 to about 3000 ng/ml, about 1500 to about 3000 ng/mL, about 1550 to about 3000 ng/mL, about 1600 to about 3000 ng/mL, about 1650 to about 3000 ng/mL, about 1700 to about 3000 ng/mL, about 1750 to about 3000 ng/ml, about 1800 to about 3000 ng/mL, about 1850 to about 3000 ng/mL, about 1900 to about 3000 ng/mL, about 1950 to about 3000 ng/mL, about 2000 to about 3000 ng/ml, about 2050 to about 3000 ng/ml, about 2100 to about 3000 ng/mL, about 2150 to about 3000 ng/mL, about 2200 to about 3000 ng/mL, about 2250 to about 3000 ng/ml, about 2300 to about 3000 ng/mL, about 2350 to about 3000 ng/mL, about 2400 to about 3000 ng/mL, about 2450 to about 3000 ng/ml, about 2500 to about 3000 ng/mL, about 2550 to about 3000 ng/ml, about 2600 to about 3000 ng/ml, about 2650 to about 3000 ng/mL, about 2700 to about 3000 ng/mL, about 2750 to about 3000 ng/ml, about 2800 to about 3000 ng/mL, about 2850 to about 3000 ng/ml, about 2900 to about 3000 ng/ml, or about 2950 to about 3000 ng/mL, including any values or ranges therebetween, following administration of the composition.

In embodiments, the oral administration of the composition provides a Cₘₐₓ of R(−)-MDMA of about 200 to about 3000 ng/mL, about 250 to about 3000 ng/mL, about 300 to about 3000 ng/mL, about 350 to about 3000 ng/mL, about 400 to about 3000 ng/ml, about 450 to about 3000 ng/mL, about 500 to about 3000 ng/mL, about 550 to about 3000 ng/ml, about 600 to about 3000 ng/ml, about 650 to about 3000 ng/mL, about 700 to about 3000 ng/ml, about 750 to about 3000 ng/mL, about 800 to about 3000 ng/mL, about 850 to about 3000 ng/mL, about 900 to about 3000 ng/mL, about 950 to about 3000 ng/ml, about 1000 to about 3000 ng/ml, about 1050 to about 3000 ng/mL, about 1100 to about 3000 ng/mL, about 1150 to about 3000 ng/ml, about 1200 to about 3000 ng/mL, about 1250 to about 3000 ng/mL, about 1300 to about 3000 ng/ml, about 1350 to about 14000 ng/mL, about 1400 to about 3000 ng/mL, about 1450 to about 3000 ng/mL, about 1500 to about 3000 ng/ml, about 1550 to about 3000 ng/ml, about 1600 to about 3000 ng/mL, about 1650 to about 3000 ng/mL, about 1700 to about 3000 ng/ml, about 1750 to about 3000 ng/mL, about 1800 to about 3000 ng/mL, about 1850 to about 3000 ng/mL, about 1900 to about 3000 ng/mL, about 1950 to about 3000 ng/mL, about 2000 to about 3000 ng/ml, about 2050 to about 3000 ng/mL, about 2100 to about 3000 ng/mL, about 2150 to about 14000 ng/ml, about 2200 to about 3000 ng/mL, about 2250 to about 3000 ng/ml, about 2300 to about 3000 ng/ml, about 2350 to about 3000 ng/mL, about 2400 to about 3000 ng/mL, about 2450 to about 3000 ng/mL, about 2500 to about 3000 ng/mL, about 2550 to about 3000 ng/ml, about 2600 to about 3000 ng/mL, about 2650 to about 3000 ng/mL, about 2700 to about 3000 ng/mL, about 2750 to about 14000 ng/ml, about 2800 to about 3000 ng/ml, about 2850 to about 3000 ng/ml, about 2900 to about 3000 ng/mL, or about 2950 to about 3000 ng/ml, including any values or ranges therebetween, following administration of the composition. In embodiments, the oral administration of the composition provides a Cₘₐₓ of R(−)-MDMA of about 225 ng/mL, about 250 ng/mL, about 275 ng/mL, about 300 ng/mL, about 325 ng/mL, about 350 ng/ml, about 375 ng/mL, about 400 ng/ml, about 425 ng/mL, about 450 ng/ml, about 475 ng/ml, about 500 ng/ml, about 525 ng/mL, about 550 ng/mL, about 575 ng/ml, about 600 ng/ml, about 625 ng/ml, about 650 ng/mL, about 675 ng/mL, about 700 ng/mL, about 725 ng/mL, about 750 ng/ml, about 775 ng/ml, about 800 ng/mL, about 825 ng/mL, about 850 ng/ml, about 875 ng/ml, about 900 ng/ml, about 925 ng/mL, about 950 ng/mL, about 975 ng/ml, about 1000 ng/mL, about 1025 ng/ml, about 1050 ng/ml, about 1075 ng/mL, about 1100 ng/mL, about 1125 ng/ml, about 1150 ng/mL, about 1175 ng/mL, about 1200 ng/ml, about 1225 ng/mL, about 1250 ng/ml, about 1275 ng/ml, about 1300 ng/mL, about 1325 ng/ml, about 1350 ng/ml, about 1375 ng/mL, about 1400 ng/ml, about 1425 ng/mL, about 1450 ng/mL, about 1475 ng/mL, about 1500 ng/mL, about 1525 ng/ml, about 1550 ng/ml, about 1575 ng/mL, about 1600 ng/mL, about 1625 ng/ml, about 1650 ng/ml, about 1675 ng/ml, about 1700 ng/ml, about 1725 ng/mL, about 1750 ng/ml, about 1775 ng/mL, about 1800 ng/mL, about 1825 ng/ml, about 1850 ng/ml, about 1875 ng/mL, about 1900 ng/ml, about 1925 ng/mL, about 1950 ng/mL, about 1975 ng/ml, about 2000 ng/mL, about 2025 ng/ml, about 2050 ng/mL, about 2075 ng/ml, about 2100 ng/ml, about 2125 ng/ml, about 2150 ng/ml, about 2175 ng/mL, about 2200 ng/mL, about 2225 ng/ml, about 2250 ng/ml, about 2275 ng/mL, about 2300 ng/ml, about 2325 ng/mL, about 2350 ng/mL, about 2375 ng/mL, about 2400 ng/ml, about 2425 ng/mL, about 2450 ng/mL, about 2475 ng/mL, about 2500 ng/mL, about 2525 ng/ml, about 2550 ng/ml, about 2575 ng/mL, about 2600 ng/mL, about 2625 ng/ml, about 2650 ng/ml, about 2675 ng/mL, about 2700 ng/mL, about 2825 ng/mL, about 2850 ng/mL, about 2875 ng/ml, to about 2900 ng/mL, about 2925 ng/mL, about 2950 ng/ml, about 2975 ng/ml, or about 3000 mg/mL, including any values or ranges therebetween, following administration of the composition. In embodiments, the dosage regimen provide a Cₘₐₓ of R(−)-MDA within the above-recited amounts. In embodiments, the dosage regimen provide a Cₘₐₓ of combined R(−)-MDMA and R(−)-MDA within the above-recited amounts.

In embodiments, the administration of the compositions of the present disclosure provides a plasma concentration ratio of R(−)-MDMA to S(+)-MDMA ranging from about 50.01:49.99 to about 100:0 (e.g., about 50.1:49.9, about 50.5:49.5, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, about 99:1, about 99.5:0.5, about 99.9:0.1, or about 100:0, among other ratios within these enumerated ratios).

In embodiments, an oral dosage form comprising from about 100 mg to about 350 mg of R(−)-MDMA, or about 125 mg to about 250 mg R(−)-MDMA, or about 150 mg to about 225 mg R(−)-MDMA provides a Cₘₐₓ of R(−)-MDMA of about 225 ng/mL, about 250 ng/ml, about 275 ng/mL, about 300 ng/mL, about 325 ng/mL, about 350 ng/mL, about 375 ng/ml, about 400 ng/ml, about 425 ng/ml, about 450 ng/mL, about 475 ng/mL, about 500 ng/mL, about 525 ng/ml, about 550 ng/ml, about 575 ng/ml, about 600 ng/ml, about 625 650 ng/ml, about 675 ng/ml, about 700 ng/ml, about 725 ng/mL, about 750 ng/mL, about 775 ng/mL, about 800 ng/ml, about 825 ng/mL, about 850 ng/ml, about 875 ng/mL, about 900 ng/mL, about 925 ng/ml, about 950 ng/ml, about 975 ng/ml, about 1000 ng/ml, about 1025 ng/ml, about 1050 ng/mL, about 1075 ng/mL, about 1100 ng/mL, about 1125 ng/mL, about 1150 ng/ml, about 1175 ng/ml, about 1200 ng/ml, about 1225 ng/ml, about 1250 ng/mL, about 1275 ng/mL, about 1300 ng/mL, about 1325 ng/ml, about 1350 ng/mL, about 1375 ng/mL, about 1400 ng/mL, about 1425 ng/ml, about 1450 ng/ml, about 1475 ng/ml, about 1500 ng/mL, about 1525 ng/mL, about 1550 ng/mL, about 1575 ng/ml, about 1600 ng/ml, about 1625 ng/ml, about 1650 ng/mL, about 1675 ng/mL, about 1700 ng/ml, about 1725 ng/ml, about 1750 ng/mL, about 1775 ng/ml, about 1800 ng/mL, about 1825 ng/ml, about 1850 ng/mL, about 1875 ng/ml, about 1900 ng/mL, about 1925 ng/ml, about 1950 ng/mL, about 1975 ng/ml, about 2000 ng/mL, about 2025 ng/mL, about 2050 ng/ml, about 2075 ng/ml, about 2100 ng/ml, about 2125 ng/mL, about 2150 ng/ml, about 2175 ng/mL, about 2200 ng/ml, about 2225 ng/ml, about 2250 ng/mL, about 2275 ng/ml, about 2300 ng/ml, about 2325 ng/ml, about 2350 ng/mL, about 2375 ng/mL, about 2400 ng/ml, about 2425 ng/ml, about 2450 ng/ml, about 2475 ng/ml, about 2500 ng/ml, about 2525 ng/ml, about 2550 ng/ml, about 2575 ng/ml, about 2600 ng/mL, about 2625 ng/ml, about 2650 ng/mL, about 2675 ng/ml, about 2700 ng/ml, about 2825 ng/mL, about 2850 ng/mL, about 2875 ng/mL, to about 2900 ng/ml, about 2925 ng/mL, about 2950 ng/mL, about 2975 ng/mL, or about 3000 mg/mL, including any values or ranges therebetween, following administration of the composition.

In embodiments, a buccal dosage form comprising from about 100 mg to about 350 mg of R(−)-MDMA, or about 125 mg to about 250 mg R(−)-MDMA, or about 150 mg to about 225 mg R(−)-MDMA provides a Cₘₐₓ of R(−)-MDMA of about 225 ng/ml, about 250 ng/ml, about 275 ng/mL, about 300 ng/mL, about 325 ng/mL, about 350 ng/mL, about 375 ng/ml, about 400 ng/mL, about 425 ng/mL, about 450 ng/mL, about 475 ng/mL, about 500 ng/ml, about 525 ng/ml, about 550 ng/ml, about 575 ng/ml, about 600 ng/mL, about 625 650 ng/mL, about 675 ng/mL, about 700 ng/mL, about 725 ng/ml, about 750 ng/mL, about 775 ng/mL, about 800 ng/ml, about 825 ng/mL, about 850 ng/ml, about 875 ng/mL, about 900 ng/ml, about 925 ng/mL, about 950 ng/ml, about 975 ng/ml, about 1000 ng/ml, about 1025 ng/ml, about 1050 ng/mL, about 1075 ng/ml, about 1100 ng/ml, about 1125 ng/ml, about 1150 ng/mL, about 1175 ng/ml, about 1200 ng/mL, about 1225 ng/ml, about 1250 ng/mL, about 1275 ng/mL, about 1300 ng/mL, about 1325 ng/ml, about 1350 ng/ml, about 1375 ng/ml, about 1400 ng/ml, about 1425 ng/mL, about 1450 ng/ml, about 1475 ng/ml, about 1500 ng/ml, about 1525 ng/ml, about 1550 ng/ml, about 1575 ng/ml, about 1600 ng/ml, about 1625 ng/mL, about 1650 ng/mL, about 1675 ng/mL, about 1700 ng/ml, about 1725 ng/ml, about 1750 ng/mL, about 1775 ng/mL, about 1800 ng/ml, about 1825 ng/ml, about 1850 ng/ml, about 1875 ng/ml, about 1900 ng/mL, about 1925 ng/ml, about 1950 ng/ml, about 1975 ng/mL, about 2000 ng/mL, about 2025 ng/mL, about 2050 ng/mL, about 2075 ng/ml, about 2100 ng/ml, about 2125 ng/ml, about 2150 ng/mL, about 2175 ng/mL, about 2200 ng/ml, about 2225 ng/mL, about 2250 ng/ml, about 2275 ng/mL, about 2300 ng/ml, about 2325 ng/ml, about 2350 ng/mL, about 2375 ng/mL, about 2400 ng/mL, about 2425 ng/mL, about 2450 ng/ml, about 2475 ng/ml, about 2500 ng/ml, about 2525 ng/ml, about 2550 ng/ml, about 2575 ng/ml, about 2600 ng/ml, about 2625 ng/mL, about 2650 ng/mL, about 2675 ng/mL, about 2700 ng/ml, about 2825 ng/ml, about 2850 ng/ml, about 2875 ng/ml, to about 2900 ng/ml, about 2925 ng/mL, about 2950 ng/ml, about 2975 ng/mL, or about 3000 mg/mL, including any values or ranges therebetween, following administration of the composition.

In embodiments, an inhaled dosage form comprising from about 100 mg to about 300 mg of R(−)-MDMA, or about 125 mg to about 250 mg R(−)-MDMA, or about 150 mg to about 225 mg R(−)-MDMA provides a Cₘₐₓ of R(−)-MDMA of about 225 ng/mL, about 250 ng/ml, about 275 ng/mL, about 300 ng/ml, about 325 ng/mL, about 350 ng/mL, about 375 ng/ml, about 400 ng/mL, about 425 ng/mL, about 450 ng/mL, about 475 ng/ml, about 500 ng/ml, about 525 ng/ml, about 550 ng/ml, about 575 ng/ml, about 600 ng/mL, about 625 650 ng/ml, about 675 ng/ml, about 700 ng/mL, about 725 ng/mL, about 750 ng/mL, about 775 ng/ml, about 800 ng/mL, about 825 ng/mL, about 850 ng/ml, about 875 ng/mL, about 900 ng/ml, about 925 ng/ml, about 950 ng/mL, about 975 ng/mL, about 1000 ng/mL, about 1025 ng/mL, about 1050 ng/ml, about 1075 ng/mL, about 1100 ng/ml, about 1125 ng/ml, about 1150 ng/ml, about 1175 ng/ml, about 1200 ng/ml, about 1225 ng/mL, about 1250 ng/mL, about 1275 ng/ml, about 1300 ng/ml, about 1325 ng/mL, about 1350 ng/ml, about 1375 ng/mL, about 1400 ng/ml, about 1425 ng/ml, about 1450 ng/mL, about 1475 ng/mL, about 1500 ng/mL, about 1525 ng/ml, about 1550 ng/ml, about 1575 ng/mL, about 1600 ng/mL, about 1625 ng/ml, about 1650 ng/ml, about 1675 ng/ml, about 1700 ng/ml, about 1725 ng/mL, about 1750 ng/mL, about 1775 ng/mL, about 1800 ng/mL, about 1825 ng/mL, about 1850 ng/mL, about 1875 ng/mL, about 1900 ng/mL, about 1925 ng/mL, about 1950 ng/mL, about 1975 ng/ml, about 2000 ng/mL, about 2025 ng/ml, about 2050 ng/mL, about 2075 ng/mL, about 2100 ng/mL, about 2125 ng/mL, about 2150 ng/mL, about 2175 ng/ml, about 2200 ng/ml, about 2225 ng/ml, about 2250 ng/mL, about 2275 ng/mL, about 2300 ng/ml, about 2325 ng/mL, about 2350 ng/mL, about 2375 ng/mL, about 2400 ng/ml, about 2425 ng/ml, about 2450 ng/mL, about 2475 ng/mL, about 2500 ng/mL, about 2525 ng/ml, about 2550 ng/ml, about 2575 ng/mL, about 2600 ng/mL, about 2625 ng/mL, about 2650 ng/ml, about 2675 ng/mL, about 2700 ng/ml, about 2825 ng/ml, about 2850 ng/ml, about 2875 ng/mL, to about 2900 ng/mL, about 2925 ng/mL, about 2950 ng/ml, about 2975 ng/mL, or about 3000 mg/mL, including any values or ranges therebetween, following administration of the composition.

In embodiments, an intranasal dosage form comprising from about 100 mg to about 350 mg of R(−)-MDMA, or about 125 mg to about 250 mg R(−)-MDMA, or about 150 mg to about 225 mg R(−)-MDMA provides a Cₘₐₓ of R(−)-MDMA of about 225 ng/ml, about 250 ng/ml, about 275 ng/mL, about 300 ng/ml, about 325 ng/ml, about 350 ng/ml, about 375 ng/ml, about 400 ng/ml, about 425 ng/ml, about 450 ng/mL, about 475 ng/mL, about 500 ng/ml, about 525 ng/ml, about 550 ng/mL, about 575 ng/mL, about 600 ng/mL, about 625 650 ng/ml, about 675 ng/ml, about 700 ng/mL, about 725 ng/mL, about 750 ng/ml, about 775 ng/mL, about 800 ng/ml, about 825 ng/ml, about 850 ng/mL, about 875 ng/ml, about 900 ng/ml, about 925 ng/mL, about 950 ng/mL, about 975 ng/mL, about 1000 ng/mL, about 1025 ng/mL, about 1050 ng/ml, about 1075 ng/mL, about 1100 ng/ml, about 1125 ng/ml, about 1150 ng/mL, about 1175 ng/mL, about 1200 ng/ml, about 1225 ng/mL, about 1250 ng/mL, about 1275 ng/ml, about 1300 ng/mL, about 1325 ng/mL, about 1350 ng/mL, about 1375 ng/mL, about 1400 ng/ml, about 1425 ng/mL, about 1450 ng/mL, about 1475 ng/mL, about 1500 ng/mL, about 1525 ng/ml, about 1550 ng/ml, about 1575 ng/ml, about 1600 ng/ml, about 1625 ng/mL, about 1650 ng/mL, about 1675 ng/ml, about 1700 ng/mL, about 1725 ng/ml, about 1750 ng/mL, about 1775 ng/mL, about 1800 ng/mL, about 1825 ng/mL, about 1850 ng/ml, about 1875 ng/mL, about 1900 ng/mL, about 1925 ng/ml, about 1950 ng/ml, about 1975 ng/mL, about 2000 ng/ml, about 2025 ng/mL, about 2050 ng/mL, about 2075 ng/mL, about 2100 ng/ml, about 2125 ng/mL, about 2150 ng/ml, about 2175 ng/ml, about 2200 ng/mL, about 2225 ng/mL, about 2250 ng/mL, about 2275 ng/mL, about 2300 ng/mL, about 2325 ng/mL, about 2350 ng/mL, about 2375 ng/mL, about 2400 ng/ml, about 2425 ng/ml, about 2450 ng/ml, about 2475 ng/mL, about 2500 ng/mL, about 2525 ng/ml, about 2550 ng/ml, about 2575 ng/mL, about 2600 ng/mL, about 2625 ng/mL, about 2650 ng/mL, about 2675 ng/ml, about 2700 ng/mL, about 2825 ng/mL, about 2850 ng/mL, about 2875 ng/mL, to about 2900 ng/ml, about 2925 ng/mL, about 2950 ng/ml, about 2975 ng/mL, or about 3000 mg/mL, including any values or ranges therebetween, following administration of the composition.

In embodiments, a parenteral dosage form comprising from about 100 mg to about 350 mg of R(−)-MDMA, or about 125 mg to about 250 mg R(−)-MDMA, or about 150 mg to about 225 mg R(−)-MDMA provides a Cₘₐₓ of R(−)-MDMA of about 225 ng/mL, about 250 ng/ml, about 275 ng/ml, about 300 ng/mL, about 325 ng/mL, about 350 ng/mL, about 375 ng/ml, about 400 ng/mL, about 425 ng/mL, about 450 ng/mL, about 475 ng/mL, about 500 ng/ml, about 525 ng/ml, about 550 ng/ml, about 575 ng/ml, about 600 ng/mL, about 625 650 ng/ml, about 675 ng/mL, about 700 ng/mL, about 725 ng/mL, about 750 ng/ml, about 775 ng/ml, about 800 ng/mL, about 825 ng/ml, about 850 ng/mL, about 875 ng/ml, about 900 ng/ml, about 925 ng/mL, about 950 ng/mL, about 975 ng/ml, about 1000 ng/mL, about 1025 ng/mL, about 1050 ng/ml, about 1075 ng/mL, about 1100 ng/mL, about 1125 ng/ml, about 1150 ng/mL, about 1175 ng/ml, about 1200 ng/mL, about 1225 ng/mL, about 1250 ng/mL, about 1275 ng/mL, about 1300 ng/ml, about 1325 ng/ml, about 1350 ng/mL, about 1375 ng/mL, about 1400 ng/ml, about 1425 ng/ml, about 1450 ng/mL, about 1475 ng/ml, about 1500 ng/mL, about 1525 ng/mL, about 1550 ng/mL, about 1575 ng/mL, about 1600 ng/mL, about 1625 ng/mL, about 1650 ng/mL, about 1675 ng/ml, about 1700 ng/mL, about 1725 ng/mL, about 1750 ng/mL, about 1775 ng/mL, about 1800 ng/ml, about 1825 ng/mL, about 1850 ng/mL, about 1875 ng/mL, about 1900 ng/ml, about 1925 ng/ml, about 1950 ng/mL, about 1975 ng/mL, about 2000 ng/mL, about 2025 ng/ml, about 2050 ng/ml, about 2075 ng/mL, about 2100 ng/mL, about 2125 ng/mL, about 2150 ng/mL, about 2175 ng/ml, about 2200 ng/mL, about 2225 ng/ml, about 2250 ng/mL, about 2275 ng/ml, about 2300 ng/ml, about 2325 ng/mL, about 2350 ng/mL, about 2375 ng/mL, about 2400 ng/ml, about 2425 ng/mL, about 2450 ng/mL, about 2475 ng/ml, about 2500 ng/ml, about 2525 ng/ml, about 2550 ng/mL, about 2575 ng/mL, about 2600 ng/mL, about 2625 ng/mL, about 2650 ng/ml, about 2675 ng/mL, about 2700 ng/mL, about 2825 ng/mL, about 2850 ng/mL, about 2875 ng/mL, to about 2900 ng/ml, about 2925 ng/mL, about 2950 ng/ml, about 2975 ng/mL, or about 3000 mg/mL, including any values or ranges therebetween, following administration of the composition.

In embodiments, a transdermal dosage form comprising from about 100 mg to about 350 mg of R(−)-MDMA, or about 125 mg to about 250 mg R(−)-MDMA, or about 150 mg to about 225 mg R(−)-MDMA provides a Cₘₐₓ of R(−)-MDMA of about 225 ng/mL, about 250 ng/mL, about 275 ng/ml, about 300 ng/mL, about 325 ng/mL, about 350 ng/mL, about 375 ng/ml, about 400 ng/mL, about 425 ng/mL, about 450 ng/mL, about 475 ng/mL, about 500 ng/mL, about 525 ng/mL, about 550 ng/ml, about 575 ng/mL, about 600 ng/mL, about 625 650 ng/ml, about 675 ng/mL, about 700 ng/mL, about 725 ng/mL, about 750 ng/ml, about 775 ng/mL, about 800 ng/ml, about 825 ng/ml, about 850 ng/ml, about 875 ng/mL, about 900 ng/mL, about 925 ng/mL, about 950 ng/mL, about 975 ng/ml, about 1000 ng/mL, about 1025 ng/mL, about 1050 ng/ml, about 1075 ng/mL, about 1100 ng/ml, about 1125 ng/mL, about 1150 ng/ml, about 1175 ng/ml, about 1200 ng/mL, about 1225 ng/mL, about 1250 ng/mL, about 1275 ng/ml, about 1300 ng/mL, about 1325 ng/mL, about 1350 ng/ml, about 1375 ng/mL, about 1400 ng/ml, about 1425 ng/mL, about 1450 ng/mL, about 1475 ng/mL, about 1500 ng/mL, about 1525 ng/ml, about 1550 ng/mL, about 1575 ng/mL, about 1600 ng/mL, about 1625 ng/ml, about 1650 ng/ml, about 1675 ng/mL, about 1700 ng/mL, about 1725 ng/mL, about 1750 ng/mL, about 1775 ng/ml, about 1800 ng/mL, about 1825 ng/ml, about 1850 ng/mL, about 1875 ng/mL, about 1900 ng/ml, about 1925 ng/ml, about 1950 ng/mL, about 1975 ng/mL, about 2000 ng/mL, about 2025 ng/ml, about 2050 ng/ml, about 2075 ng/ml, about 2100 ng/mL, about 2125 ng/ml, about 2150 ng/mL, about 2175 ng/mL, about 2200 ng/mL, about 2225 ng/mL, about 2250 ng/mL, about 2275 ng/ml, about 2300 ng/ml, about 2325 ng/mL, about 2350 ng/mL, about 2375 ng/mL, about 2400 ng/ml, about 2425 ng/ml, about 2450 ng/ml, about 2475 ng/mL, about 2500 ng/mL, about 2525 ng/mL, about 2550 ng/ml, about 2575 ng/mL, about 2600 ng/ml, about 2625 ng/mL, about 2650 ng/ml, about 2675 ng/ml, about 2700 ng/mL, about 2825 ng/mL, about 2850 ng/mL, about 2875 ng/ml, to about 2900 ng/ml, about 2925 ng/ml, about 2950 ng/ml, about 2975 ng/mL, or about 3000 mg/mL, including any values or ranges therebetween, following administration of the composition.

In embodiments, the administration of the compositions of the present disclosure provide a Tₘₐₓ of R(−)-MDMA of about 1 hour to about 10 hours (such as about 2 hours to about 8 hours, about 3 hours to about 8 hours, or about 3 hours to about 5 hours), for example, about 1 hour, 1.25 hours, about 1.5 hours, about 1.75 hours, about 2.0 hours, about 2.25 hours, about 2.5 hours, about 2.75 hours, about 3.0 hours, about 3.25 hours, about 3.5 hours, about 3.75 hours, about 4.0 hours, about 4.25 hours, about 4.5 hours, about 4.75 hours, about 5.0 hours, about 5.25 hours, about 5.5 hours, about 5.75 hours, about 6.0 hours, about 6.25 hours, about 6.5 hours, about 6.75 hours, about 7.0 hours, about 7.25 hours, about 7.5 hours, about 7.75 hours, about 8.0 hours, about 8.25 hours, about 8.5 hours, about 8.75 hours, about 9.0 hours, about 9.25 hours, about 9.5 hours, about 9.75 hours, or about 10 hours, including any values or ranges therebetween following administration of the composition. In embodiments, the administration of the composition of the present disclosure provide a Tₘₐₓ of R(−)-MDMA of about 3 hour to about 8 hours, wherein the amount of composition of the present disclosure comprises about 175 to 225 mg MDMA free base or an equivalent dose of the pharmaceutically acceptable salt or prodrug thereof.

In embodiments, the administration of the compositions of the present disclosure provide a T_{1/2} of R(−)-MDMA of about 5 hour to about 20 hours, for example, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, or about 20 hours, including any values or ranges therebetween following administration of the composition. In embodiments, the administration of the composition of the present disclosure provide a T_{1/2} of R(−)-MDMA of about 7 hour to about 18 hours, wherein the amount of composition of the present disclosure comprises about 175 to 225 mg MDMA free base or an equivalent dose of the pharmaceutically acceptable salt or prodrug thereof.

In embodiments, the administration of the compositions of the present disclosure provide a Tₘₐₓ of R(−)-MDA of about 5 hours to about 30 hours (such as about 10 hours to about 24 hours), for example, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, or about 15 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 24.5 hours, or about 25 hours, about 26 hours, about 26.5 hours, about 27 hours, about 27.5 hours, about 28 hours, about 28.5 hours, about 29 hours, about 29.5 hours, or about 30 hours, including any values or ranges therebetween following administration of the composition.

In embodiments, the administration of the composition provides a AUC₀₋ₜ of R(−)-MDMA of about 2000 ng·h/mL to about 20,000 ng·h/mL, for example, about 2000 ng·h/mL, about 3000 ng·h/mL, about 4000 ng·h/mL, about 5000 ng·h/mL, about 6000 ng·h/mL, about 7000 ng·h/mL, about 8000 ng·h/mL, about 9000 ng·h/mL, about 10,000 ng·h/mL, about 11,000 ng·h/mL, about 12,000 ng·h/mL, about 13,000 ng·h/mL, about 14,000 ng·h/mL, about 15,000 ng·h/mL, 16,000 ng·h/mL, about 17,000 ng·h/mL, about 18,000 ng·h/mL, about 19,000 ng·h/mL, or about 20,000 ng·h/mL, including any values or ranges therebetween, following administration of the composition.

In embodiments, the administration of the composition provides a AUC₀₋ₜ of R(−)-MDA of about 100 ng·h/mL to about 800 ng·h/mL, for example, about 100 ng·h/mL, about 150 ng·h/mL, about 200 ng·h/mL, about 250 ng·h/mL, about 300 ng·h/mL, about 350 ng·h/mL, about 400 ng·h/mL, about 450 ng·h/mL, about 500 ng·h/mL, about 550 ng·h/mL, about 600 ng·h/mL, about 650 ng·h/mL, about 700 ng·h/mL, about 750 ng·h/mL, about 800 ng·h/mL, including any values or ranges therebetween, following administration of the composition.

### IV. Methods and Uses

In aspects, the present disclosure provides methods used to treat a neurological disease, disorder, or condition, (inclusive of, e.g., a mental health disease, disorder, or condition). The methods comprise administering a composition of the disclosure to a subject in need thereof.

As referred to herein, a “neurological disease disorder, or condition” refers to a disease, disorder, or condition that can comprise: a neuropsychiatric disorder, such as depression (including severe depression such as treatment-resistant depression (TRD), major depressive disorder (MDD) and persistent depressive disorder), alexithymia, catatonic depression, a depressive disorder due to a medical condition, postpartum depression, premenstrual dysphoric disorder, seasonal affective disorder, anxiety, anxiety disorder, social anxiety disorder (SAD), general anxiety disorder (GAD), avolition disorder, bipolar disorder (including bipolar I disorder and bipolar II disorder), post-traumatic stress disorder (PTSD), Cluster C personality disorders, rejection sensitivity dysphoria/disorder, body dysmorphic disorder, abnormalities of mood or emotion, including the above conditions, dysthymia, schizoaffective disorder, schizophrenia and other psychotic disorders, panic disorder, traumatic stress disorders, phobic disorders, and personality disorders with abnormal mood, such as borderline personality disorder, schizoid and schizotypal disorders, suicide ideation, rumination/unproductive repetitive thoughts negatively impacting one's behavior, mood, and/or ability to focus, obsessive-compulsive disorder, addiction (including substance use disorders, such as addiction to nicotine, alcohol, cocaine, opioids, amphetamine, methamphetamine, heroin, morphine, phencyclidine, 3,4-methylenedioxy-methamphetamine, as well as other addictive substances), addictive behavior (including eating, gambling, sex, pornography, videogames, work, exercise, spiritual obsession, self-harm, travel and shopping addiction, etc.), eating disorders (including anorexia nervosa, bulimia nervosa and binge eating disorder), and pain (including pain associated with migraine or headache or chronic pain).

In embodiments, the disclosure relates to the use of the compositions for treating or alleviating one or more neurological diseases, disorders, or conditions. In embodiments, the neurological disease, disorder, or condition comprises a mental health disease, disorder, or condition. Such conditions include those identified herein, which are described and discussed in the Diagnostic and Statistical Manual of Mental Disorders (5^{th} ed.; DSM-5; American Psychiatric Association, 2013), the content of which is incorporated by reference in its entirety for any purpose.

In embodiments, the disclosure provides methods for the treatment of an anxiety disorder. In embodiments, the anxiety disorder is one or more of generalized anxiety disorder (GAD), phobia, social anxiety disorder (SAD), social phobia, specific phobia, panic disorder, panic attack, panic attach specifier, separation anxiety disorder, selective mutism, agoraphobia, situation anxiety or an anxiety disorder induced by a substance/medication or due to another medical condition. Anxiety disorders are described and discussed in Diagnostic and Statistical Manual of Mental Disorders, Fifth Edition, Text Revision (DSM-5-TR), the content of which is incorporated by reference in its entirety for any purpose. In embodiments, the methods reduce at least one symptom of anxiety disorder in the subject, such as fear, anxiety, separation anxiety, and/or panic attacks (e.g. frequency and severity of attacks). In embodiments, the methods reduce other behavioral symptoms such as inattentiveness, hyperactivity, and/or impulsiveness. In embodiments, the methods improve clinical scores described herein in the subject.

In embodiments, the disclosure provides methods for the treatment of social anxiety disorder (SAD) comprises administering a therapeutically effective amount of the composition of the present disclosure to a subject in need thereof. SAD is a marked fear or anxiety about one or more social situations and is interchangeable with “social phobia.” SAD is characterized by the persistent fear of social or performance situations in which embarrassment may occur, such as parties, meetings, eating in front of others, writing in front of others, public speaking, conversations, meeting new people, and other related situations. Exposure to social or performance situations provokes an immediate anxiety response, as well as sweating, trembling, racing or pounding heartbeat, mental confusion, and a desire to flee. Social avoidance and isolation can also become extreme, especially in the more generalized condition. A generalized sub-type, also referred to as performance-based SAD, is a type of social anxiety disorder where people experience intense fear and anxiety when performing or speaking in public, but not in other social situations. In embodiments, the method of the present disclosure is more effective in a subject having SAD that is not a performance only sub-type. The criteria for diagnosis of SAD are described and discussed in DSM-5, the content of which is incorporated by reference in its entirety for any purpose.

In embodiments, the subject with SAD has one or more comorbidities. Non-limiting examples of comorbidities include anxiety disorder, depressive disorder, psychotic disorder, developmental disorder, eating disorder, attention-deficit hyperactivity disorder (ADHD), attention deficit disorder (ADD), autism spectrum disorder (ASD), COVID-19, disorders associated with COVID-19, tic disorder, bipolar disorder, conduct disorder, mental retardation, or a combination thereof. In embodiments, the comorbidity is a psychiatric disorder selected from schizophrenia, schizoaffective disorder, bipolar disorder, major depressive disorder, anxiety disorder, obsessive compulsive disorder, post-traumatic stress disorder, attention deficit hyperactivity disorder, autism, alcohol use disorder, drug use disorder, or suicide attempt. In embodiments, the comorbidity is a mood disorder, an anxious distress, a rejection sensitivity, a mixed anxiety and depressive disorder (MADD), a major depressive disorder, a bipolar disorder, schizophrenia, an eating disorder, attention deficit/hyperactivity disorder, epilepsy, a cardiovascular disease, post-traumatic stress disorder (PTSD), migraine, irritable bowel syndrome, dementia, Alzheimer's disease, Parkinson's disease, or combinations thereof. In embodiments, the comorbidity is depression, generalized anxiety, agoraphobia, or panic disorder. In embodiments, the comorbidity is an anxiety disorder, a mood disorder, an impulse control disorder, a substance use disorder, or any disorder. In embodiments, the comorbidity is hypertension, dyslipidemias, sleep problems/disorders, pain, functional gastrointestinal symptoms/disorders, menstrual dysfunction, pregnancy complications, intracranial hypertension, polycystic ovary syndrome, or combinations thereof.

In embodiments, the disclosure provides methods for the treatment of generalized anxiety disorder (GAD). GAD is characterized by excessive anxiety and worry, fatigue, restlessness, increased muscle aches or soreness, impaired concentration, irritability, and/or difficulty sleeping.

In embodiments, the subject with a GAD has one or more comorbidities. Non-limiting examples of comorbidities include anxiety disorder, depressive disorder, psychotic disorder, developmental disorder, eating disorder, attention-deficit hyperactivity disorder (ADHD), COVID-19, disorders associated with COVID-19, attention deficit disorder (ADD), autism spectrum disorder (ASD), tic disorder, bipolar disorder, conduct disorder, mental retardation, or a combination thereof. In embodiments, the comorbidity is a mood disorder, major depressive disorder, bipolar disorder, schizophrenia, an eating disorder, attention deficit/hyperactivity disorder, epilepsy, cardiovascular disease, migraine, irritable bowel syndrome, dementia, Alzheimer's disease, Parkinson's disease, or combinations thereof. In embodiments, the comorbidity is depression, generalized anxiety, agoraphobia, or panic disorder. In some embodiments, the comorbidity is major depression. In embodiments, the comorbidity is an eating disorder. In some embodiments, the comorbidity is ADHD. In embodiments, the comorbidity is ASD. In some embodiments, the comorbidity is COVID-19, disorders associated with COVID-19, or both. Non-limiting examples of disorders associated with COVID-19 include post-traumatic stress disorder, anxiety disorder, acute stress disorder, adjustment disorder, or panic disorder. In embodiments, the comorbidity is major depressive disorder, bipolar disorder, irritable bowel disease, or irritable bowel syndrome.

In embodiments, the disclosure provides methods for the treatment of depression, substance use disorders (SUD), and/or eating disorders. In embodiments, the disclosure provides methods for the treatment of an eating or feeding disorder, such as, for example anorexia nervosa, bulimia nervosa, binge eating, and/or other disorders related to eating.

In embodiments, the disclosure provides methods for the treatment of a mood disorder such as, for example, depressive disorders, such as major depressive disorder or treatment resistant depression.

In embodiments, the method can treat a dissociative disorder such as depersonalization-derealization disorder.

In embodiments, the method can treat a somatic symptom disorder and related disorders.

In embodiments, the method can treat a feeding and eating disorder such as anorexia nervosa, bulimia nervosa, and/or binge-eating disorder.

In embodiments, the method can treat a condition relating to sexual dysfunction such as, for example, female orgasmic disorder, female sexual interest disorder, female arousal disorder, and/or male hypoactive sexual desire disorder.

In embodiments, the method can treat a disruptive, impulse-control, and/or conduct disorder. In embodiments, the disruptive, impulse-control, and conduct disorder comprises antisocial personality disorder and/or a non-substance related disorder such as gambling disorder.

In embodiments, the method can treat a personality disorder, such as disorders classified as Cluster A, Cluster B, and/or Cluster C disorders which include, for example, adjustment disorder, antisocial personality disorder, borderline personality disorder, histrionic personality disorder and/or avoidant personality disorder.

In embodiments, the method can treat a depressive episode with short-duration hypomania.

In embodiments, the method can treat a disorder relating to non-suicidal self-injury.

In embodiments, the disclosure provides methods for the treatment of a substance abuse disorder. In embodiments, substance use related disorders are typically disorders of maladaptive patterns of substance use, and include criteria, such as recurrent substance use related problems, tolerance to a substance, withdrawal upon discontinuing use, an inability to cut down or control use of the substance, and giving up important social, occupational, or recreational activities because of using the substance. See e.g., DSM-V. In embodiments, the substance use related disorder is a disorder resulting from the use of: alcohol; caffeine; cannabis; hallucinogens (such as phencyclidine or similarly acting arylcyclohexylamines, and other hallucinogens, such as LSD); inhalants; opioids; sedatives, hypnotics, or anxiolytics; stimulants (including amphetamine-type substances, cocaine, and other stimulants); tobacco; and other substances.

In embodiments, the disclosure provides methods for the treatment of one or more conditions, or associated symptoms, comprising anosognosia (e.g., denial of deficit or lack of insight), borderline personality disorder, bereavement, rejection sensitivity (e.g., as associated with attention deficit disorders and/or hyperactivity disorders), and Cluster C personality disorders, (e.g., avoidant personality disorder, dependent personality disorder, and/or obsessive-compulsive personality disorder), which are typically characterized as including a consistently dysfunctional pattern of anxious thinking or behavior.

In embodiments, a subject who has, develops, or is suspected of having or developing, a Cluster C personality disorder can be diagnosed, identified, and/or characterized as also exhibiting symptoms of at least one other type of Cluster C disorder. As such, the number of symptoms exhibited by a subject may vary widely.

Avoidant personality disorder can be associated with symptoms such as, for example, high or increased sensitivity to criticism or rejection; feelings of inadequacy, lack of importance, and/or unattractive; isolation; withdrawn from activities and/or social interaction; fear of disapproval; fear of embarrassment; and/or a target of criticism.

Dependent personality disorder can be associated with symptoms such as, for example, a lack of self-reliance; overly-reliant on others; being submissive and/or clingy toward others; fear of abandonment; lack of self-confidence; inability to make decisions without input from others; inability to motivate or self-start; seeking approval from others; endures abuse or poor treatment from others; and/or relationship seeking.

Obsessive-compulsive personality disorder (different from obsessive-compulsive disorder, which is an anxiety-based disorder) can be associated with symptoms such as, for example, over-emphasis on details, orderliness and/or rules; seeking perfection; controlling of people, tasks, money, situations and circumstances; inability to delegate tasks; rigidity in attitude; neglecting relationships (i.e., favoring work over friends); stubbornness; and/or inflexibility in attitude toward alternative opinions and/or perspectives.

Borderline personality disorder can be associated with symptoms such as, for example, difficulty processing or managing emotion, self-image issues, highly variable, quick, intense, and/or extreme mood and/or behavioral changes, impulsive behavior (spending, promiscuity, binge eating and/or drinking, recklessness, etc.), self-harming behavior (suicidal thoughts, threats, and/or attempts), avoidance of abandonment, intense and/or unstable relationships, lack of trusting in others, feelings of emptiness, inability to manage anger, and/or feelings of dissociation (e.g., dissociated from one's body).

In embodiments relating to adjustment disorders, the disorder can be characterized by the development of emotional and/or behavioral symptoms in response to an identifiable stressor within three months of symptom onset. The type of stressor may vary in significance for children and adolescents relative to adults. The symptoms or behaviors are usually clinically significant, typically including one or both of: (i) marked distress that is out of proportion to the severity or intensity of the stressor (given external context and the cultural factors that might influence symptom severity and presentation); and, (ii) significant impairment in social, occupational, and/or other important areas of functioning. Adjustment disorders are typically triggered by a stress-related disturbance that does not meet any criteria for other mental disorders, or a heightening of a preexisting condition. The symptoms associated with an adjustment disorder are also typically not characteristic of normal bereavement. In addition, once the stressor has ended, the symptoms do not persist for more than an additional six months. Adjustment disorders can be characterized as acute (lasting less than six months) or chronic (lasting more than six months). Typically adjustment disorders can be identified by one or more of six general subtypes, including (a) depressed mood; (b) anxiety; (c) mixed anxiety and depressed mood; (d) disturbance of conduct; (e) mixed disturbance of emotions and conduct; and (f) unspecified (not classifiable as one of the specific subtypes (a)-(e)). These six types of adjustment disorders can be characterized by several symptoms such as those described below.

Adjustment disorder with depressed mood: Symptoms can include feeling sad, tearful and hopeless, very tired, and taking no pleasure in the things the subject enjoyed previously.

Adjustment disorder with anxiety: Symptoms can include nervousness, worry, having a hard time concentrating or remembering things, and/or feeling overwhelmed. Children may strongly fear being separated from their parents and loved ones.

Adjustment disorder with mixed anxiety and depressed mood: Symptoms can include a blend of depression and anxiety.

Adjustment disorder with disturbed conduct: Symptoms can include behavioral problems, such as fighting or reckless driving. Children and teenagers may skip school or damage or destroy property.

Adjustment disorder with disturbed emotions and conduct: Symptoms can include a mix of depression, anxiety and behavioral problems.

Unspecified adjustment disorder: Symptoms do not fit well within the other types of adjustment disorders, but can include physical problems, problems in relationships with family or friends, or problems at work or school.

In embodiments, the compositions, uses, and methods treat a subject who has or develops a disease, disorder, or condition that is associated with anosognosia (lack of insight or denial of deficit). In such embodiments, the methods and uses can treat a subject who is unable to recognize the underlying illness or condition and/or may not seek treatment or maintain an existing treatment. Anosognosia can be associated with many mental health disorders, but is most often associated in subjects who have or develop schizophrenia, dementia, Alzheimer's disease, and bipolar disorder, as non-limiting examples. Non-limiting symptoms of anosognosia include an inability to: recognize one's own illness or medical problem; recognize the signs and symptoms of the condition being experienced; to connect signs and symptoms being experienced with the underlying disorder or condition; and/or understand or agree that treatment is needed for the underlying disorder or that the condition is serious.

In embodiments, the disclosed methods are effective to treat one or more disease, disorder, or condition that is characterized by one or more symptoms. In embodiments, the one or more symptoms can be one or more transdiagnostic symptom(s) that are associated with more than one disease, disorder, or condition. In embodiments, the method in accordance with the disclosure can treat the underlying disease, disorder, or condition and/or treat the transdiagnostic symptom(s) (e.g., reducing severity, intensity, frequency of the symptom(s), blocking the symptom(s), slowing progression or escalation of the symptom(s), reducing or eliminating recurrence of the symptom(s), inhibiting the symptom(s), or ameliorating the symptom(s), etc.). In embodiments, the transdiagnostic symptom comprises any one or more of symptoms including, for example, one or more symptoms associated with a general increase associated with rejection sensitivity, emotional regulation, anhedonia, self-doubt/criticism, and/or lack of insight. In embodiments, the one or more transdiagnostic symptom comprises, cognitive inflexibility or a lack of ability to shift perspective, negative emotional attention bias, a tendency to focus attention on negatively-valenced emotions and/or events, cognitive dysregulation, intrusive thoughts, restricted empathy for ones-self, restricted empathy for others, rigid thinking, non-response to psychotherapy, distorted cognitions, inappropriate responses, negative self-appraisal, negative feelings of self-worth, anxiety, rumination, depressed mood, negative cognitions, loss of ability to feel pleasure, social withdrawal or avoidance, problems with interpersonal relationships, diminished pleasure, loss of libido, emotional bluntedness, lack of emotional regulation, irritability, anger, mood swings, intense outburst, aggressive outburst, self-harm, suicidal ideation, substance abuse, lack of impulse control, and/or fear from an expectation of rejection (whether real or imagined).

### V. Combination Therapies and Psychological Support

In embodiments, the methods of the disclosure can comprise administering a second therapy, such as, for example, one or more agents (e.g., selective serotonin reuptake inhibitors (SSRIs)) such as those typically administered for the treatment of neurological, psychological, and/or mental health conditions such as major depressive disorder, Cluster C personality disorders, borderline personality disorder, and anxiety disorders, and the like. Accordingly, the disclosure provide methods for R(−)-MDMA-assisted psychotherapy. In embodiments, the disclosure provide methods that improve and/or expand target indications for MDMA-assisted psychotherapy, by administering an R(−)-MDMA composition of the disclosure in methods of R(−)-MDMA-assisted psychotherapy.

In embodiments, a second therapy comprises a form of psychotherapy such as, for example, cognitive processing therapy (CPT), cognitive behavioral therapy (CBT), dialectical behavioral therapy (DBT), exposure therapy (ET), brief eclectic psychotherapy (BEP), narrative exposure therapy (NAT), or eye-movement desensitization, reprocessing (EMDR), or digital technology-based psychotherapy (e.g., MoodGYM, MindSpot, or text messaging).

In embodiments, the second therapy comprises an antidepressant, an anticonvulsant, lisdexamfetamine dimesylate, an antipsychotic, an anxiolytic, an anti-inflammatory drug, a benzodiazepine, an analgesic drug, a cardiovascular drug, an opioid antagonist, or combinations thereof.

In embodiments, the second therapy is an antipsychotic. In embodiments, the antipsychotic is a phenothiazine, butyrophenone, thioxanthene, clozapine, risperidone, olanzapine, or sertindole, quetiapine, aripiprazole, zotepine, perospirone, a neurokinin-3 antagonist, such as osanetant and talnetant, rimonabant, or a combination thereof.

In embodiments, administration of the second therapy and/or therapeutic is prior to administration of the compositions described herein. In embodiments, administration of the second therapy and/or therapeutic is after administration of the compositions described herein. In embodiments, administration of the second therapy and/or therapeutic is concurrent with administration of the compositions described herein.

In embodiments, the administration of the composition described herein is in combination with a psychological support. Psychological support, also called emotional support, includes, but not limited to, trained mental healthcare professional support, community and family support activities, schooling, activating social networks, biological, emotional, spiritual support, cultural and social support, psychological safety management, art activities, and sports activities, and theatric activities.

### VI. Formulations

In embodiments, the pharmaceutical compositions can be formulated in a variety of unit dosage forms depending upon the method and route of administration. In embodiments, the methods can include any route of administration such as, for example, oral, parenteral (intravenous (i.v.), intraperitoneal (i.p.), intramuscular, (i.m.), intrathecal, or subcutaneous, (s.c.) injections), sublingual, buccal, rectal, vaginal, ocular, otic, nasal, inhalation, nebulization, cutaneous/topical, or transdermal. Suitable unit dosage forms, can include, but are not limited to liquids, powders, tablets, pills, capsules, lozenges, sprays, granules, etc., and can also include conventional non-toxic pharmaceutically acceptable carriers, adjuvants and vehicles and, if desired, other active ingredients. As discussed herein, and as known in the art, the term parenteral includes percutaneous, subcutaneous, intravascular (e.g., intravenous), intramuscular, or intrathecal injection or infusion techniques and the like.

In embodiments, such pharmaceutical compositions may be in a form suitable for oral use, for example, as tablets, troches, lozenges, aqueous or oily suspensions, dispersible powders or granules, emulsion, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide palatable preparations. Tablets contain the active ingredient (i.e., R(−)-MDMA enantiomer(s)) in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the disclosure may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative, and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compositions of the disclosure may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

### VII. Clinical Outcome Measures

In embodiments, the methods of the disclosure provide reductions/ameliorations in at least one symptom of social anxiety disorder (SAD) or at least one scale item related to SAD as measured by any scores described herein or known to those skilled in the art. In embodiments, the reductions/ameliorations in at least one symptom of social anxiety disorder (SAD) can be measured using any one of the assessments described herein. In general, symptoms of SAD include physical symptoms (e.g., blushing, sweating, trembling, rapid heartbeat, chest pain, nausea, dizziness, shortness of breath, and light-headedness), behavioral symptoms (e.g., rigid body posture, overly soft voice, difficulty making eye contact, avoiding eye contact, low self-esteem, and appearing unfriendly, aloof, or arrogant), cognitive symptoms (e.g., feeling like your mind is going blank, self-consciousness, fear of being judged negatively, and fear that others will notice you look anxious), avoidance symptoms (e.g., avoiding places with other people, avoiding doing things or speaking to people, avoiding situations where you might be the center of attention, and declining invitations), and/or anticipatory symptoms (anxiety in anticipation of a feared activity or event).

In embodiments, the methods of the disclosure provide reductions/ameliorations in at least one symptom of generalized anxiety disorder (GAD) or at least one scale item related to GAD as measured by any scores described herein or known to those skilled in the art. In embodiments, the reductions/ameliorations in at least one symptom of GAD can be measured using any one of the assessments described herein. In general, symptoms of GAD include mental symptoms (e.g., feeling apprehensive, restless, or keyed up, having trouble concentrating, or feeling like your mind goes blank), physical symptoms (e.g., muscle tension, headaches, stomachaches, or other unexplained pains, difficulty swallowing, trembling or twitching, sweating, hot flashes, or lightheadedness), trouble falling or staying asleep, or restless nights, difficulty handling uncertainty, indecisiveness and fear of making the wrong decision, inability to let go of worries, startling easily, and/or frequent need to use the bathroom.

In embodiments, the efficacy of the methods of the disclosure is measured through procedures, methodologies, or techniques for assessing social anxiety known to those skilled in the art. In embodiments, clinical efficacies are measured by one or more of the following: Liebowitz Social Anxiety Scale (LSAS) total score and sub-scale scores, Clinical Global Impression (CGI)-Severity of Illness scale, CGI-Improvement of Illness scale, Social Phobia Inventory (SPIN) total score, social Connectedness Scale, Internalized Shame Scale, Quick Inventory of Depressive Symptomatology-16 Items (QIDS-SR-16), Subtle Avoidance Frequency Examination (SAFE), Short Form-36 (SF-36) total score, Columbia Suicide Severity Rating Scale, Self-Compassion Scale (SCS) total score, Challenging Experience Questionnaire (CEQ) total score, Spoken Intensity Rating Scale (SIRS), Emotional Breakthrough Inventory (EBI) total score, Social Self-Compassion Scale (SSCS) total score, social self-efficacy scale (SSES), 5-Dimensions-Altered States of Consciousness (5D-ASC), 11-Dimensions-Altered States of Consciousness (11D-ASC), Mystical Experience Questionnaire, EuroQol-5 Dimension (EQL-5D), Hamilton Rating Scale for Anxiety (HAM-A), Hamilton Rating Scale for Depression (HAM-D), axis V Social and Occupational Functioning Assessment Scale of DSM-IV, axis II (ICD-10) World Health Organization Disability Assessment, Schedule 2 (DAS-2), Sheehan Disability Scales, Schneier Disability Profile, World Health Organization Quality of Life-100 (WHOQOL-100), Quality of Life Inventory (QOLI), Pediatric Anxiety Rating Scale (PARS), European Quality of Life 5-Dimensions scale, State-Trait Anxiety Inventory (STAI), Difficulties in Emotional Regulation Scale (DERS), Emotional Regulation Questionnaire (ERQ-10), GAD-7, State Self-Esteem Scale (SSES), Rejection Sensitivity Questionnaire-Adult, Inventory of Interpersonal Problems (IIP), Beck Cognitive Insight Scale (BCIS), Positive and Negative Affect Schedule (PANAS-SF), Behavior Rating Inventory of Executive Function (BRIEF), Cognitive Flexibility Inventory (CFI), Acceptance and Action Questionnaire (AAQ/AAQ-II), Beck Anxiety Inventory (BAI), Psychological Insight Scale (PIS-6) total score, Hospital Anxiety and Depression Scale (HADS), Generalized Anxiety Disorder questionnaire-IV (GADQ-IV), Hamilton Anxiety Rating Scale (HARS), Overall Anxiety Severity and Impairment Scale (OASIS), Hospital Anxiety and Depression Scale (HADS), Subject Health Questionnaire 4 (PHQ-4), Brief Trauma Questionnaire (BTQ), Combat Exposure Scale (CES), Mississippi Scale for Combat-Related PTSD (M-PTSD), Posttraumatic Maladaptive Beliefs Scale (PMBS), Perceived Threat Scale (DRRI-2 Section: G), PTSD Symptom Scale-Interview for DSM-5 (PSS-I-5), Structured Interview for PTSD (SI-PTSD), Davidson Trauma Scale (DTS), Impact of Event Scale-Revised (IES-R), Posttraumatic Diagnostic Scale (PDS-5), Potential Stressful Events Interview (PSEI), Stressful Life Events Screening Questionnaire (SLESQ), Spielberger's Trait and Anxiety, Generalized Anxiety Disorder 7-Item Scale, The Psychiatric Institute Trichotillomania Scale (PITS), The MGH Hairpulling Scale (MGH-HPS), The NIMH Trichotillomania Severity Scale (NIMH-TSS), The NIMH Trichotillomania Impairment Scale (NIMH-TIS), The Clinical Global Impression (CGI), the Brief Social Phobia Scale (BSPS), The Panic Attack Questionnaire (PAQ), Panic Disorder Severity Scale, Florida Obsessive-Compulsive Inventory (FOCI), The Leyton Obsessional Inventory Survey Form, The Vancouver Obsessional Compulsive Inventory (VOCI), The Schedule of Compulsions, Obsessions, and Pathological Impulses (SCOPI), Padua Inventory-Revised (PI-R), Quality of Life (QoL), The Clinical Global Improvement (CGI) scale, The Yale-Brown Obsessive-Compulsive Scale (Y-BOCS), The Yale-Brown Obsessive-Compulsive Scale Second Edition (Y-BOCS-II), The Dimensional Yale-Brown Obsessive-Compulsive Scale (DY-BOCS), The National Institute of Mental Health-Global Obsessive-Compulsive Scale (NIMH-GOCS), The Yale-Brown Obsessive-Compulsive Scale Self-Report (Y-BOCS-SR), The Obsessive-Compulsive Inventory-Revised (OCI-R), and the Dimensional Obsessive-Compulsive Scale (DOCS), or a combination thereof. While the above non-exhaustive list of scores are typical of those that might be used, other procedures, methodologies, or techniques known to those skilled in the art may alternatively be used.

In embodiments, the clinical outcomes are measured as a baseline prior to the administration, and it continues to be monitored throughout the administration period. In embodiments, a subject in need thereof has clinician-administered Liebowitz Social Anxiety Scale (LSAS) total score of at least about 55, at least abut 60, at least about 65, or at least about 70 prior to the administration. In embodiments, a subject in need thereof has SAD as measured by clinician-administered LSAS total score of at least about 70 prior to the administration. In embodiments, a subject in need thereof has CGI-Severity score of at least about 4, at least about 5, or at least about 6 prior to the administration.

In embodiments, the methods of the present disclosure provide a reduction in the subject's LSAS total score by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, compared to prior to administration. In embodiments, the methods of the present disclosure provide a reduction in the subject's LSAS total score by at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, or more, compared to prior to administration. In embodiments, the methods of the present disclosure provides remission as measured by the subject's LSAS total score of less than 30.

In embodiments, the methods of the present disclosure provide a reduction in the subject's CGI-Improvement score or CGI-severity score by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, compared to prior to administration. In embodiments, the methods of the present disclosure provide a reduction in the subject's CGI-Improvement score or CGI-severity score by at least about 1, at least about 2, at least about 3, at least about 4, at least about 5, at least about 6, or at least about 7, compared to prior to administration.

In embodiments, the methods of the present disclosure provide a reduction in the subject's total and sub-scale scores on the 5D-ASC rating by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, compared to prior to administration. In embodiments, the methods of the present disclosure provide a reduction in the subject's total and sub-scale scores on the 5D-ASC rating by at least about 10, at least about 20, at least about 30, at least about 40, at least about 50, at least about 60, or at least about 70, compared to prior to administration.

In embodiments, the methods of the present disclosure provide a reduction in the subject's sub-scale scores on the Challenging Experience Questionnaire (CEQ) by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, compared to prior to administration. In embodiments, the methods of the present disclosure provide a reduction in the subject's sub-scale scores on the CEQ rating scale by at least about 10, at least about 20, at least about 30, at least about 40, at least about 50, at least about 60, or at least about 70, compared to prior to administration.

In embodiments, the methods of the present disclosure provide an increase in the subject's Spoken Intensity Rating Scale (SIRS) by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, compared to prior to administration. In embodiments, the methods of the present disclosure provide an increase in the subject's SIRS by at least about 1, at least about 2, at least about 3, at least about 4, at least about 5, at least about 6, or at least about 7, compared to prior to administration.

In embodiments, the methods of the present disclosure provide an increase in the subject's Self-reported Patient Global Impressions of Change (PGI-C) score by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, compared to prior to administration. In embodiments, the methods of the present disclosure provide an increase in the subject's PGI-C score by at least about 1, at least about 2, at least about 3, at least about 4, at least about 5, or at least about 6, compared to prior to administration.

In embodiments, the methods of the present disclosure provide a reduction in the subject's total scores and sub-scale scores on the self-reported Social Phobia Inventory (SPIN) by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, compared to prior to administration. In embodiments, the methods of the present disclosure provide a reduction in the subject's total scores and sub-scale scores on the SPIN by at least about 10, at least about 15, at least about 20, at least about 25, at least about 30, at least about 35, at least about 40, at least about 45, at least about 50, at least about 55, or at least about 60, compared to prior to administration.

In embodiments, the methods of the present disclosure provide a reduction in the subject's Quick Inventory of Depressive Symptomatology (Self Report) 16 item (QIDS-SR-16) total score by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, compared to prior to administration. In embodiments, the methods of the present disclosure provide a reduction in the subject's QIDS-SR-16 total score by at least about 5, at least about 10, at least about 15, at least about 20, or at least about 25, compared to prior to administration.

In embodiments, the methods of the present disclosure provide a reduction in the subject's Subtle Avoidance Frequency Examination (SAFE) total score by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, compared to prior to administration. In embodiments, the methods of the present disclosure provide a reduction in the subject's SAFE total score by at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, compared to prior to administration.

In embodiments, after administration of the composition described herein, at least one symptom of social anxiety disorder is alleviated for a period of at least about 5 minutes to at least about 1 year, for example, at least one symptom of anxiety is alleviated for a period of at least about 5 minutes, at least about 15 minutes, at least about 30 minutes, at least about 45 minutes, at least about 1 hour, at least about 2 hours, at least 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 2 months, at least about 3 months, at least about 4 months, at least about 5 months, at least about 6 months, at least about 7 months, at least about 8 months, at least about 9 months, at least about 10 months, at least about 11 months, at least about 12 months, or more. In embodiments, at least one symptom of anxiety is alleviated for a period of at least 1 month after administration of the composition described herein. In embodiments, at least one symptom of anxiety is alleviated for a period of at least 3 months after administration of the composition described herein. In embodiments, at least one symptom of anxiety is alleviated for a period of at least 1 year after administration of the composition described herein.

### Numbered Embodiments of the Disclosure

In addition to the disclosure above, the Examples below, and the appended claims, the disclosure sets forth the following numbered embodiments.

1. A method of treating an anxiety disorder in a subject in need thereof, comprising administering to the subject a composition comprising an enantiomerically enriched form of R(−)-3,4-methylenedioxymethamphetamine (R(−)-MDMA), or a pharmaceutically acceptable salt or prodrug thereof,
wherein the administration provides a R(−)-MDMA Tₘₐₓ of about 2 hours to about 10 hours and a R(−)-MDMA Cₘₐₓ of about 150 ng/mL to about 3000 ng/mL following administration.
   2. The method of embodiment 1, wherein the anxiety disorder is social anxiety disorder.
   3. The method of embodiment 1, wherein the anxiety disorder is generalized anxiety disorder.
   4. The method of any one of embodiments 1-3, wherein the subject has one or more conditions comorbid with the anxiety disorder.
   5. The method of embodiment 4, wherein the one or more conditions comorbid with an anxiety disorder is a mood disorder, a depressive disorder, an anxious distress, a rejection sensitivity, a mixed anxiety and depressive disorder (MADD), a major depressive disorder, or a treatment resistant depression, a bipolar disorder, schizophrenia, an eating disorder, an attention deficit/hyperactivity disorder, epilepsy, a cardiovascular disease, a substance abuse disorder, migraine, a headache disorder, an irritable bowel syndrome, a dementia, post-traumatic stress disorder (PTSD), Alzheimer's disease, Parkinson's disease or any combination thereof.
   6. The method of embodiment 5, wherein the eating disorder comprises anorexia nervosa, bulimia nervosa, or binge eating.
   7. The method of embodiment 5, wherein the substance abuse disorder comprises alcohol abuse, caffeine abuse, cannabis abuse, hallucinogen abuse, inhalant abuse, opioid abuse, sedative abuse, hypnotic abuse, anxiolytic abuse, stimulant abuse, or tobacco abuse.
   8. The method of embodiment 7, wherein the hallucinogen is selected from phencyclidine or similarly acting arylcyclohexylamines, and lysergic acid diethylamide (LSD).
   9. The method of embodiment 7, wherein the stimulant is selected from amphetamine-type substances and cocaine.
   10. The method of any one of embodiments 1-9, wherein about 75 mg to about 350 mg R(−)-MDMA free base or an equivalent dose of the pharmaceutically acceptable salt or prodrug thereof is administered to the subject.
   11. The method of any one of embodiments 1-10, wherein about 75 mg to about 225 mg R(−)-MDMA free base or an equivalent dose of the pharmaceutically acceptable salt or prodrug thereof is administered to the subject.
   12. The method of any one of embodiments 1-10, wherein about 75 mg, about 125 mg, about 175 mg, or about 225 mg R(−)-MDMA free base or an equivalent dose of the pharmaceutically acceptable salt or prodrug thereof is administered to the subject.
   13. The method of any one of embodiments 1-10, wherein about 225 mg R(−)-MDMA free base or an equivalent dose of the pharmaceutically acceptable salt or prodrug thereof is administered to the subject.
   14. The method of any one of embodiments 1-13, wherein the subject is administered 2 repeated doses of the composition at an interval of about 14 days to about 56 days.
   15. The method of any one of embodiments 1-14, wherein the subject is administered 2 repeated doses of the composition at an interval of about 28 days.
   16. The method of any one of embodiments 1-13, wherein the subject is administered once about every about 2 weeks to about 6 months.
   17. The method of any one of embodiments 1-16, wherein administering the composition comprises injection, oral delivery, transdermal delivery, or transmucosal delivery.
   18. The method of embodiment 17, wherein the composition is orally administered.
   19. The method of any one of embodiments 1-18, further comprising administering a second therapy.
   20. The method of embodiment 19, wherein the second therapy comprises a psychological support.
   21. The method of any one of the preceding embodiments, wherein the composition comprises a weight ratio of R(−)-MDMA to S(+)-MDMA ranging from about 50.1:49.9 to about 100:0.
   22. The method of the preceding embodiments, wherein the composition comprises R(−)-MDMA in an enantiomerically enriched form of ≥90%, ≥95%, ≥96%, ≥97%, ≥98%, ≥99%, ≥99.5%, ≥99.9%, or ≥99.99%, or 100% relative to total MDMA.
   23. The method of any one of the preceding embodiments, wherein the composition comprises R(−)-MDMA in an enantiomeric excess to S(+)-MDMA from about 95% to about 100%.
   24. The method of any one of the preceding embodiments, wherein the composition comprises R(−)-MDMA in an enantiomeric excess to S(+)-MDMA from about 98% to about 100%.
   25. The method of any one of the preceding embodiments, wherein the administration provides a R(−)-MDMA Tₘₐₓ of about 3 hours to about 8 hours.
   26. The method of any one of the preceding embodiments, wherein the administration provides a R(−)-MDMA Tₘₐₓ of about 3 hour to about 5 hours.
   27. The method of any one of the preceding embodiments, wherein the administration provides a R(−)-MDMA AUC₀₋ₜ of about 2000 ng·h/mL to about 20,000 ng·h/mL following administration.
   28. The method of any one of the preceding embodiments, wherein the subject has SAD that is not a performance only sub-type.
   29. The method of any one of the preceding embodiments, wherein the subject has a Liebowitz Social Anxiety Scale (LSAS) total score ≥70 prior to the administration.
   30. The method of embodiment 29, wherein the administration provides a reduction in the subject's LSAS total score by at least about 20 compared to prior to the administration.
   31. The method of embodiment 29, wherein the subject's LSAS total score is reduced to no greater than 50 after the administration.
   32. The method of embodiment 29, wherein the administration provides a reduction in the subject's LSAS sub-scale score by at least about 10% compared to prior to the administration.
   33. The method of any one of the preceding embodiments, wherein the subject has Clinician Global Impressions-Severity (CGI-S) score ≥4 prior to the administration.
   34. The method of any one of the preceding embodiments, wherein the administration provides a reduction in the subject's Clinician-rated Clinical Global Impressions-Improvement (CGI-I) score by at least about 10% compared to prior to the administration.
   35. The method of any one of the preceding embodiments, wherein the administration provides a reduction in the subject's total and sub-scale scores on the 5D-ASC rating scale by at least about 10% compared to prior to the administration.
   36. The method of any one of the preceding embodiments, wherein the administration provides a reduction in the subject's sub-scale scores on the Challenging Experience Questionnaire (CEQ) by at least about 10% compared to prior to the administration.
   37. The method of any one of the preceding embodiments, wherein the administration provides an increase in the subject's Spoken Intensity Rating Scale (SIRS) by at least about 10% compared to prior to the administration.
   38. The method of any one of the preceding embodiments, wherein the administration provides an increase in the subject's Self-reported Patient Global Impressions of Change (PGI-C) score by at least about 10% compared to prior to the administration.
   39. The method of any one of the preceding embodiments, wherein the administration provides a reduction in the subject's self-reported Social Phobia Inventory (SPIN) total score and sub-scale scores by at least about 10% compared to prior to the administration.
   40. The method of any one of the preceding embodiments, wherein the administration provides a reduction in the subject's Quick Inventory of Depressive Symptomatology (Self Report) 16 item (QIDS-SR-16) total score by at least about 10% compared to prior to the administration.
   41. The method of any one of the preceding embodiments, wherein the administration provides a reduction in the subject's Subtle Avoidance Frequency Examination (SAFE) total score by at least about 10% compared to prior to the administration.

### EXAMPLES

The following examples are provided to further illustrate the embodiments of the present disclosure but are not intended to limit the scope of the claims. While they are typical of those that might be used, other procedures, methodologies, or techniques known to those skilled in the art may alternatively be used.

### Example 1: R(−)-MDMA Nonclinical Studies

Nonclinical studies were conducted to elucidate the therapeutic potential of R(−)-MDMA. These studies included in vitro assessments of agonist activity at human serotonin receptor 5-HT₂ receptor subtypes and neurite outgrowth in rat cortical neuron cultures. In vivo studies in mice evaluated the ability of R(−)-MDMA to induce the 5-HT_{2A} receptor agonism-mediated HTR and facilitate extinction of fear learning, a model relevant to human exposure-based therapies used in the treatment of SAD and other anxiety disorders.

### In Vitro Agonism at Human 5-HT2 Receptor Subtypes

To evaluate agonist activity of R(−)-MDMA, human 5-HT_{2A}, 5-HT_{2B}, and 5-HT_{2C} (non-edited and edited) receptor G-protein-dependent inositol monophosphate accumulation was evaluated in Chinese hamster ovaries (CHO)-K1 recombinant cell lines using homogeneous time-resolved fluorescence assays. As 5-HT_{2C} receptor RNA may undergo editing, allowing for the existence of multiple receptor isoforms that show differences in G protein coupling efficiency, functional activity of R(−)-MDMA was assessed at the non-edited 5-HT_{2C} receptor and an edited 5-HT_{2C} receptor variant. The agonism of R(−)-MDMA at each 5-HT2 receptor subtype was assessed at concentrations ranging from 0.003 to 100 μM, and the EC50 value was determined for each receptor. Agonist efficacy of R(−)-MDMA was expressed as a percentage of the activity of the reference agonist, α-methyl-serotonin (α-Me-5-HT), at its EC100 concentration.

Table 1 presents the agonist potency (EC50) values for R(−)-MDMA and racemic MDMA across human 5-HT2 receptor subtypes, as well as efficacy at each receptor relative to α-Me-5-HT. In these assays, R(−)-MDMA demonstrated moderate potency partial agonism at 5-HT_{2A} receptors and high potency full agonism at 5-HT_{2B} and 5-HT_{2C} (non-edited and edited) receptors.

TABLE 1
EC50 values for R(—)-MDMA and racemic MDMA

| | R(—)-MDMA | | Racemic MDMA | |
|---|---|---|---|---|
| | | Efficacy | | Efficacy |
| | EC₅₀ | (% of a-Me-5- | EC₅₀ | (% of a-Me-5- |
| Receptor | (μM) | HT response) | (μM) | HT response) |
| 5-HT_{2A} | 3.342 | 69.3 | 4.728 | 68.4 |
| 5-HT_{2B} | 0.053 | 91.8 | 0.194 | 91.2 |
| 5-HT_{2C} (non-edited) | 0.376 | 97.9 | 0.943 | 95.3 |
| 5-HT_{2C} (edited) | 0.494 | 96.6 | 0.881 | 97.7 |

### In Vitro Rat Cortical Neurite Outgrowth

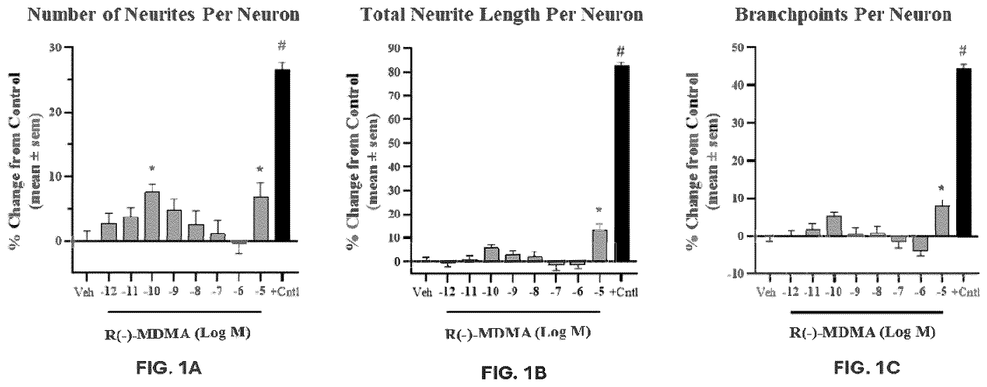

To evaluate the potential for structural neuroplasticity-like effects, which may underlie learning and memory and counter the effects of stress, R(−)-MDMA was evaluated on measures of neurite outgrowth in rat embryonic cortical neurons following exposure for 3 days at each of 8 concentrations, ranging from 0.001 nM to 10 μM. The experiment included a 0.1% sterile water vehicle negative control condition and a 250 nM donepezil positive control condition, and the number of neurites per neuron, total neurite length per neuron and branchpoints per neuron from n=12 wells per treatment were compared to the vehicle control condition. Donepezil significantly and robustly increased each of the 3 structural neuroplasticity measures compared to vehicle, expressed as a percentage of the vehicle control response, demonstrating the ability of the assay to detect neurite outgrowth-promoting activity (FIGS. 1A-1C). R(−)-MDMA induced modest yet significant increases in the number of neurites per neuron at 0.0001 and 10 μM (FIG. 1A) as well as total neurite length (FIG. 1B) and branchpoints per neuron (FIG. 1C) at 10 μM. These results indicate that R(−)-MDMA may promote neurite outgrowth, a form of structural neuroplasticity that may play a role in adaptive learning processes involved in SAD therapy and counter the pathophysiological effects of stress.

### Mouse Head Twitch Response

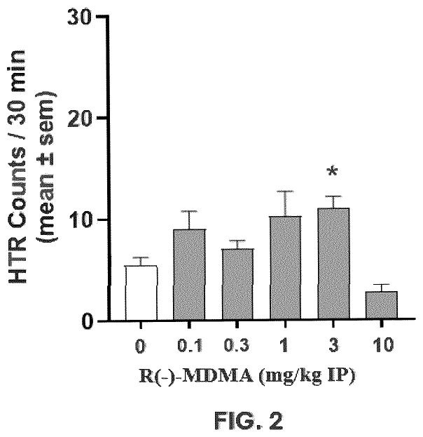

Mouse Head Twitch (HTR) is a side-to-side rotational head movement in rodents that is widely employed as a behavioral measure of 5-HT_{2A} receptor activation (Halberstadt and Geyer 2013). The present study was designed to objectively investigate dose-dependent effects of R(−)-MDMA on the mouse HTR. Animals with head mounted magnets were dosed intraperitoneally and immediately tested for HTR for 30 min using an automated magnetometer detection system (Halberstadt and Geyer 2013). R(−)-MDMA significantly increased HTR at 3 mg/kg compared to the vehicle control group (FIG. 2). These results indicate that R(−)-MDMA exhibits 5-HT_{2A} receptor agonism mediated activity in vivo.

### Mouse Extinction of Fear Learning Assay

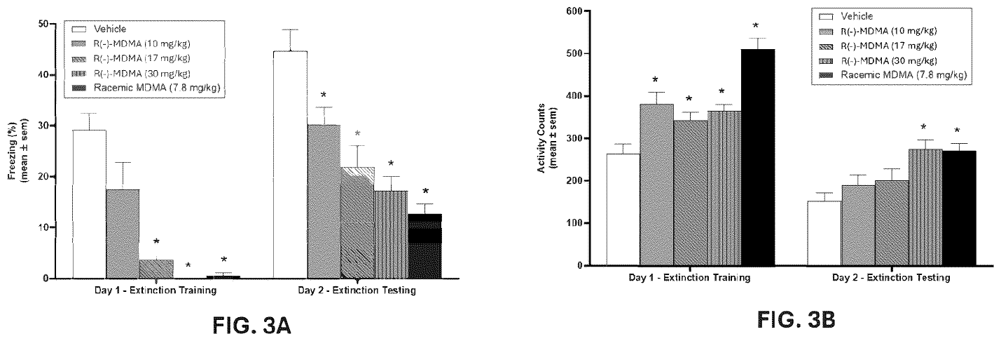

Extinction of fear learning in rodents is a useful assay to understand if compounds have the potential to enhance learning processes that may be involved in recovery from SAD and other stress and anxiety related disorders, due to its resemblance to exposure-based therapy in humans. To confirm therapeutic potential, the present study was conducted to evaluate the effects of a single administration of R(−)-MDMA in a mouse model of fear extinction using a procedure modified from Young et al., 2015. Vehicle (0.9% saline), R(−)-MDMA (10, 17 or 30 mg/kg) or the reference control racemic MDMA (7.8 mg/kg) was administered IP, once, 30 min prior to extinction training (Day 1). Freezing behavior, an indicator of the fear response, was measured in the presence of the CS during the extinction training session (Day 1) and the day after (Day 2, Extinction Testing, in the absence of test article). Locomotor activity was measured during the initial 2-minute habituation period, prior to the presentation of the CS, on Days 1 and 2. Compared to vehicle, the reference control, racemic MDMA (7.8 mg/kg), significantly reduced freezing behavior on Days 1 and 2 (FIG. 3A) and increased locomotor activity on Days 1 and 2 (FIG. 3B). R(−)-MDMA (17 and 30 mg/kg) exhibited significant reductions of freezing behavior on Days 1 and 2 (FIG. 3A), while 10 mg/kg R(−)-MDMA significantly reduced freezing behavior on Day 2 only (FIG. 3B). R(−)-MDMA (30 mg/kg) also exhibited significant increases in activity counts on Days 1 and 2 (FIG. 3B), while 10 and 17 mg/kg R(−)-MDMA significantly increased activity on Day 1 only (FIG. 3B). The results indicate that R(−)-MDMA may enhance learning processes that may be involved in recovery from SAD.

### Example 2: R(−)-MDMA Phase 1 Clinical Study

A Phase 1 clinical study was conducted to evaluate the effects of orally administered R(−)-MDMA in healthy volunteers. 32 healthy participants (mean age of 28.5 years (range from 19-47 years); 58.3% female, 41.7% male) enrolled in a four-cohort, single-ascending dose, randomized, double-blind, placebo-controlled study. The cohorts (all fasted, with the exception of one arm) received a single dose of 75 mg, 125 mg, 175 mg or 225 mg of R(−)-MDMA or placebo in a 6+2 design.

Enantiomerically selective R(−)-MDMA (98.8% R(−)-MDMA HCl by achiral HPLC; 100.0% R(−)-MDMA HCl by chiral chromatography) was prepared as powder (API) in capsules (HPMC capsule shells) at two strengths, 29.7 mg drug substance (corresponding to 25 mg of R(−)-MDMA free base) and 118.9 mg drug substance (corresponding to 100 mg of R(−)-MDMA free base).

The primary endpoints assessed the safety, tolerability, pharmacokinetics (PK), and exploratory pharmacodynamic (PD) observations. Treatment-related adverse events (AEs) were in line with expectations (e.g., nausea at 29.2%, headache at 16.7%) and generally dose dependent. There were no study discontinuations, and no serious or severe AEs were observed. Non-clinically significant increases in blood pressure and heart rate were observed, though such changes showed limited dose dependency. Peak body temperatures fell within the normal range. Bruxism was observed in a single subject at the highest dose (225 mg).

### Pharmacokinetics (PK)

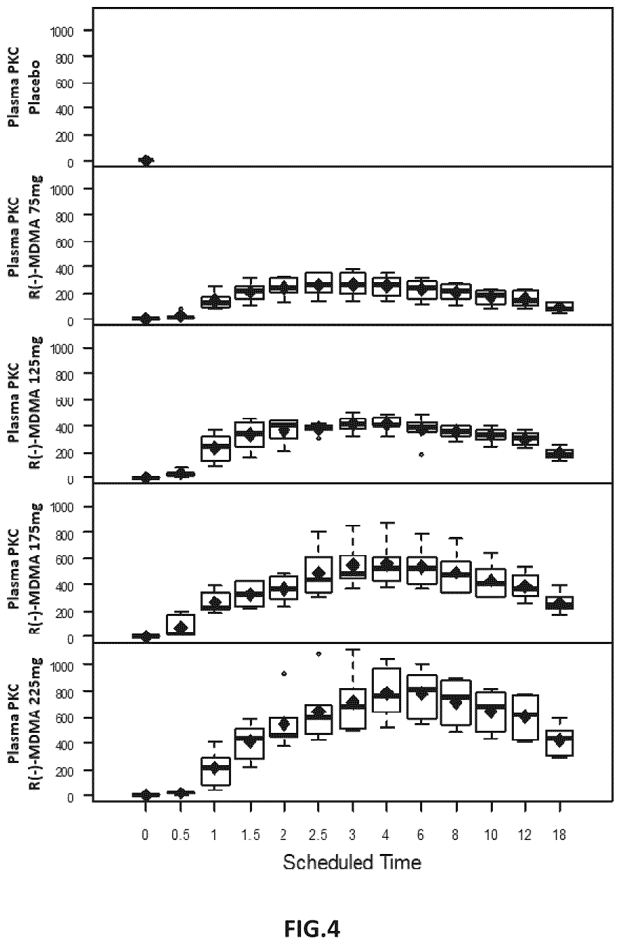

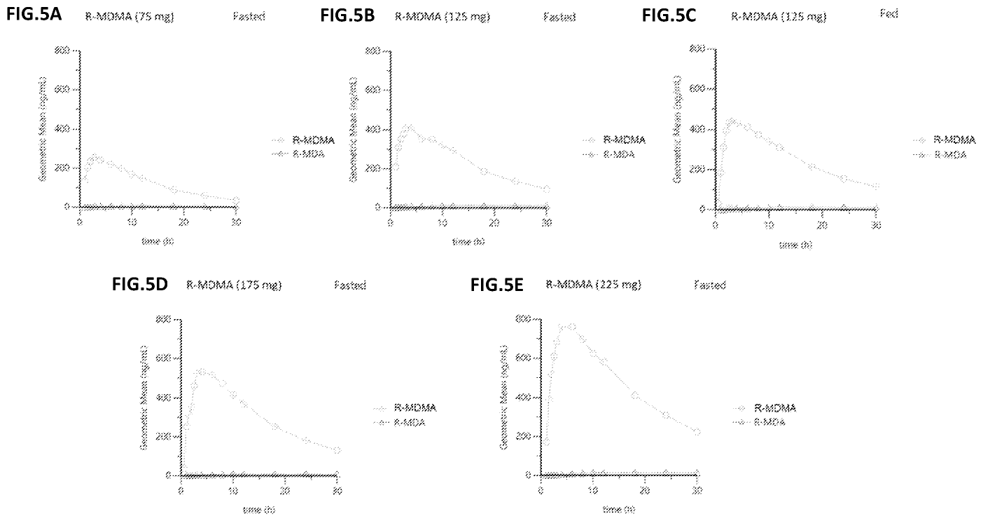

A summary of the plasma PK parameters in the Phase 1 clinical study is provided in Table 2. Following oral administration of a single dose of 75 to 225 mg, R(−)-MDMA was rapidly absorbed at all dose levels and exposure and Cₘₐₓ were increased with increasing dose levels. Plasma concentrations, in ng/mL, for each cohort (placebo, 75 mg, 125 mg (fasted and fed), 175 mg, 225 mg) as a function of time are presented in FIG. 4. FIGS. 5A-5E show the geometric means of plasma concentrations of R(−)-MDMA and its metabolite R(−)-MDA for each cohort. Mean plasma R-MDA peaked at approximately 12 hours postdose. Dose proportionality was not established for plasma R(−)-MDMA or R-MDA. No food effect was concluded, indicating that R(−)-MDMA may be administered with or without food.

Mean AUC (AUC₀₋ₜ, AUC₀₋₂₄, AUC₀₋₃₀, AUC_{0-inf}) and Cₘₐₓ were increased with increasing dose. Mean dose-normalized AUC₀₋ₜ, AUC₀₋₃₀, and AUC_{0-inf} showed an increase across the dose groups, suggesting a slightly greater than dose proportional increase in exposure which is consistent with previous non-clinical studies in rats. In contrast, mean dose-normalized Cₘₐₓ was generally similar across cohorts. Both the median Tₘₐₓ (ranging from 3 to 5 hours) and the mean t_{1/2} (ranging from 9 to 14 hours) increased with dose level. Furthermore, in dose proportionality analyses for plasma R(−)-MDMA Cₘₐₓ, AUC₀₋ₜ and AUC_{0-inf}, the 90% CI did not fall within the bounds of dose proportionality (0.797 to 1.203) and proportionality was therefore not established. Mean total clearance (CL/F) was highest in the R(−)-MDMA 75 mg group and mean apparent volume of distribution (Vz/F) generally decreased with increasing dose. Mean metabolite: parent ratio of AUC (MR_{AUC}) was comparable at all dose levels. The mean plasma concentration of R(−)-MDMA remained above the lower limit of quantitation (LLOQ) at the final PK assessments at 30 hours postdose.

TABLE 2
Pharmacokinetic Data

| | | R(—)-MDMA | | R-MDA | |
|---|---|---|---|---|---|
| Treatment group | Parameter | n | Value ^{a} | n | Value ^{a} |

Day 1

| | | | | | |
|---|---|---|---|---|---|
| R(—)-MDMA | Cₘₐₓ (ng/mL) | 6 | 275.8217 (90.7390) | 6 | 7.6733 (1.6139) |
| 75 mg | Tₘₐₓ (hr) | 6 | 3.000 (2.02, 3.00) | | 12.000 (10.00, 12.00) |
| (N = 6) | AUC₀₋ₜ (h · ng/mL) | 6 | 3976.7452 (1350.4178) | 6 | 183.2765 (40.5712) |
| | t_{1/2} (hr) | 6 | 9.2622 (2.5227) | 6 | 39.0473 (19.0557) |
| | MR_{AUC} | 6 | 0.0490 (0.0123) | — | — |
| R(—)-MDMA | Cₘₐₓ (ng/mL) | 6 | 425.2550 (62.4569) | 6 | 12.7950 (3.1122) |
| 125 mg | Tₘₐₓ (hr) | 6 | 3.517 (1.50, 4.02) | | 12.025 (12.00, 30.03) |
| (N = 6) | AUC₀₋ₜ (h · ng/mL) | 6 | 7135.8809 (1245.2963) | 6 | 307.4702 (66.2889) |
| | t_{1/2} (hr) | 6 | 11.8615 (1.9947) | — | NC |
| | MR_{AUC} | 6 | 0.0443 (0.0122) | — | — |
| R(—)-MDMA | Cₘₐₓ (ng/mL) | 5 | 617.3440 (158.9596) | 5 | 11.2820 (2.1914) |
| 175 mg | Tₘₐₓ (hr) | 5 | 4.000 (3.00, 6.02) | | 18.000 (12.00, 18.00) |
| (N = 6) | AUC₀₋ₜ (h · ng/mL) | 5 | 10007.8882 (2324.8851) | 5 | 272.6761 (57.5614) |
| | t_{1/2} (hr) | 5 | 12.2009 (1.4988) | — | NC |
| | MR_{AUC} | 5 | 0.0287 (0.0108) | — | — |
| R(—)-MDMA | Cₘₐₓ (ng/mL) | 6 | 828.6450 (213.1377) | 6 | 17.4733 8.0424 |
| 225 mg | Tₘₐₓ (hr) | 6 | 5.000 (3.00, 6.02) | | 21.050 (18.00, 30.00) |
| (N = 6) | AUC₀₋ₜ (h · ng/mL) | 6 | 14453.1811 (3684.7488) | 6 | 413.2388 (193.5541) |
| | t_{1/2} (hr) | 6 | 14.0772 (3.2340) | — | NC |
| | MR_{AUC} | 6 | 0.0299 (0.0144) | — | — |

Day 9

| | | | | | |
|---|---|---|---|---|---|
| R(—)-MDMA | Cₘₐₓ (ng/mL) | 6 | 455.4850 (65.0461) | 6 | 13.3733 (2.3199) |
| 125 mg | Tₘₐₓ (hr) | 6 | 2.758 (2.08, 4.02) | | 12.042 (12.00, 30.00) |
| (N = 6) | AUC₀₋ₜ (h · ng/mL) | 6 | 7229.6028 (1863.6711) | 6 | 291.6730 (112.2988) |
| | t_{1/2} (hr) | 6 | 13.3444 (3.5309) | 2 | 143.1218 (145.7332) |
| | MR_{AUC} | 6 | 0.0404 (0.0156) | — | — |

Abbreviations: AUC_{0-inf} = area under the drug concentration-time curve, from time 0 (time of dosing or start of the infusion) extrapolated to infinity; AUC₀₋ₜ = area under the drug concentration-time curve, from time 0 (time of dosing or start of the infusion) to the last measurable concentration; Cₘₐₓ = maximum observed plasma concentration; MR_{AUC} = Metabolic ratio (AUC_{0-inf} R-MDA/AUC_{0-inf} R(—)-MDMA); SD = standard deviation; t_{1/2} = apparent terminal half-life; Tₘₐₓ = time to first maximum observed concentration.
^{a}Pharmacokinetic values are reported as mean (SD) for Cₘₐₓ, AUC_{0-24 h}, t_{1/2}, and MR_{AUC}, and as median (minimum, maximum) for Tₘₐₓ.

R(−)-MDMA was detected in urine in all participants by the first collection interval (0-4 hours postdose). Mean urine R(−)-MDMA concentration was highest in the 12-24 hour collection interval, after which it rapidly decreased in the 24-30 hour collection interval. The mean cumulative amount excreted in urine (CAe) of R(−)-MDMA excreted generally showed a dose-related increase. The mean renal clearance (CLr) was approximately similar across all dose groups. No clear trend was observed in mean fraction of the administered dose excreted into the urine (CFe) among the dose levels. The urinalysis results of the phase I study are summarized in Table 3.

TABLE 3
Urinalysis Results

| | | R(—)-MDMA | | R-MDA | |
|---|---|---|---|---|---|
| Treatment group | Parameter | n | Value ^{a} | n | Value ^{a} |

Day 1

| | | | | | |
|---|---|---|---|---|---|
| R(—)-MDMA | Ae₁₂₋₂₄ (mg) | 6 | 7.6917 (3.1509) | 6 | 0.4622 (0.1101) |
| 75 mg | CLr (L/h) | 6 | 4.9966 (1.2255) | 6 | 4.6787 (0.8471) |
| (N = 6) | CAe (mg) | 6 | 20.0733 (9.7605) | 6 | 0.8510 (0.2149) |
| | Fe₁₂₋₂₄ (%) | 6 | 10.2556 (4.2012) | 6 | 0.6163 (0.1468) |
| | CFe (%) | 6 | 26.7643 (13.0140) | 6 | 1.1347 (0.2865) |
| R(—)-MDMA | Ae₁₂₋₂₄ (mg) | 6 | 14.5039 (5.6733) | 6 | 0.7850 (0.4299) |
| 125 mg | CLr (L/h) | 6 | 3.9900 (1.0288) | 6 | 3.7173 (0.9547) |
| (N = 6) | CAe (mg) | 6 | 28.2063 (7.8540) | 6 | 1.1628 (0.4509) |
| | Fe₁₂₋₂₄ (%) | 6 | 11.6031 (4.5387) | 6 | 0.6280 (0.3440) |
| | CFe (%) | 6 | 22.5650 (6.2832) | 6 | 0.9302 (0.3608) |
| R(—)-MDMA | Ae₁₂₋₂₄ (mg) | 5 | 20.3702 (13.5536) | 5 | 0.7076 (0.4510) |
| 175 mg | CLr (L/h) | 5 | 5.9108 (2.5782) | 4 | 4.3945 (2.1019) |
| (N = 6) | CAe (mg) | 5 | 59.6271 (34.2201) | 5 | 1.4788 (0.8740) |
| | Fe₁₂₋₂₄ (%) | 5 | 11.6401 (7.7449) | 5 | 0.4043 (0.2577) |
| | CFe (%) | 5 | 34.0726 (19.5543) | 5 | 0.8450 (0.4995) |
| R(—)-MDMA | Ae₁₂₋₂₄ (mg) | 6 | 36.8357 (16.8611) | 6 | 0.8450 (0.4995) |
| 225 mg | CLr (L/h) | 6 | 5.5411 (1.6424) | 6 | 5.1337 (2.2871) |
| (N = 6) | CAe (mg) | 6 | 76.0465 (15.1167) | 6 | 1.9805 (0.8173) |
| | Fe₁₂₋₂₄ (%) | 6 | 16.3714 (7.4938) | 6 | 0.8802 (0.3633) |
| | CFe (%) | 6 | 33.7985 (6.7186) | 6 | 0.5432 (0.2708) |

Day 9

| | | | | |
|---|---|---|---|---|
| EMP-01 | Ae₁₂₋₂₄ (mg) | 6 | 18.5655 (12.7511) | 0.9794 (0.8552) |
| 125 mg | CLr (L/h) | 6 | 5.2324 (1.8093) | 5.3793 (1.5400) |
| (N = 6) | CAe (mg) | 6 | 40.5567 (15.5851) | 0.9794 (0.8552) |
| | Fe₁₂₋₂₄ (%) | 6 | 14.8524 (10.2009) | 0.7835 (0.6842) |
| | CFe (%) | 6 | 32.4453 (12.4681) | 1.2746 (0.7265) |

Abbreviations: Ae₁₂₋₂₄ = Amount excreted at the 12-24 hour collection interval; CAe = Cumulative amount of drug excreted; CFe = Cumulative fraction of drug excreted; CLr = Renal clearance rate; Fe₁₂₋₂₄ = The fraction of drug excreted at the 12-24 hour collection interval.

### Exploratory Pharmacodynamics (PD)

### Neuroplasticity Outcomes

Mature BDNF (mBDNF) is a biomarker of neuroplasticity, which has been shown to correlate with improved efficacy with classic psychedelics. Increases in neuroplasticity may counter the pathophysiological effects of anxiety and stress on the brain via neural network growth and enhancing the ability for learning/retention of new social threat-assessment thresholds (De Vos et al., 2021. Psychedelics and neuroplasticity: a systematic review unraveling the biological underpinnings of psychedelics, Frontiers in psychiatry. 12: 724606). In the phase I study, there was a numerical increase in mBDNF at Day +1 in the R(−)-MDMA 225 mg group only. All other cohorts, numerical decreases in mBDNF were found. A nominal reduction in the mean concentration of mBDNF was observed on Day 2 in both the R(−)-MDMA Total and pooled placebo groups. A nominal increase in the mean concentration of mBDNF was observed on Day 10 in the R(−)-MDMA 125 mg group in the fed state, in contrast to a decrease in the fasted state; however, individual changes from Baseline were variable.

### Spoken Intensity Rating Scale (SIRS) Results

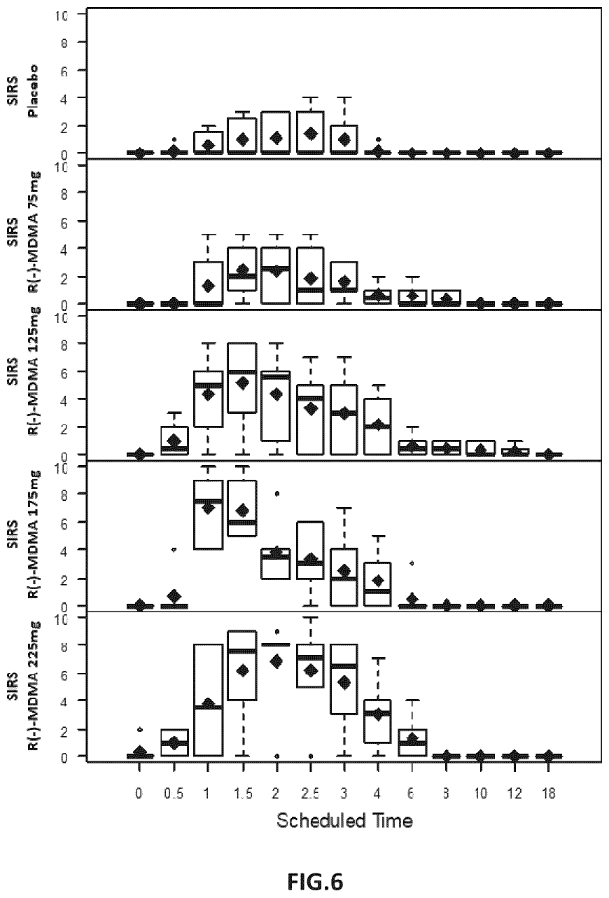

The SIRS results showed a strong dose response and that the peak SIRS occurred shortly after detectable increases in R(−)-MDMA plasma concentration, but much earlier than Tₘₐₓ at each dose (FIG. 6). Neurobehavioral assessment results in the fasted state showed small nominal increases from Baseline in mean spoken intensity rating scale (SIRS) scores in each cohort at 1 to 2 hours postdose. Beyond 8 hours postdose, mean SIRS scores remained ≤0.5 in all cohorts. The dose-dependent increase in SIRS indicates that R(−)-MDMA may has 5-HT2A serotonergic psychedelic-like characteristics. This is unexpected given the prior results reported for racemic MDMA (Parrott A C., Human psychopharmacology of Ecstasy (MDMA): a review of 15 years of empirical research. Hum Psychopharmacol. 2001 December; 16 (8): 557-577).

### Exploratory Visual Analog Scale (VAS) Results

VAS measure changes in subjective experience that are typical in healthy human volunteer studies. These include experiences such as Happy, Open, Loving, Close to Others, Sociable, and Lonely. In the phase I study, R(−)-MDMA increased VAS scores compared to placebo for positive affect, in the domains of happy, loving, openness, and less loneliness, and in a dose-dependent pattern. A clear dose response was observed for “openness” in the higher R(−)-MDMA dose groups.

### Hormone Response—Prolactin and Oxytocin

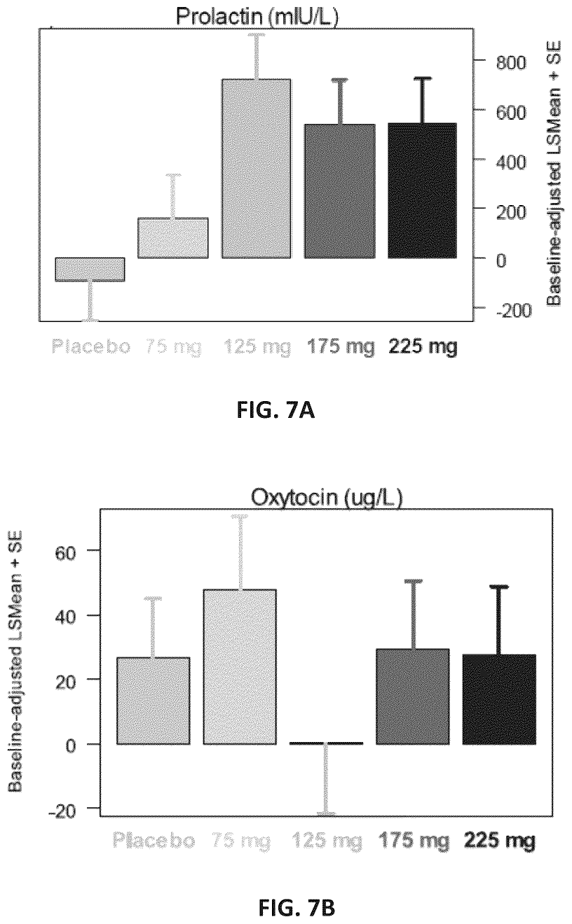

Among the hormones measured among the cohorts, a consistent statistically significant increase in the concentration of prolactin was observed in response to R(−)-MDMA dose levels at 125 mg-225 mg compared with the placebo group (FIG. 7A). This suggests involvement of serotonergic signaling, as increases in prolactin can be mediated by 5-HT release. In contrast, R(−)-MDMA did not appear to have an effect on oxytocin production in any of the cohorts when compared with the placebo group. As illustrated in FIG. 7B, none of the R(−)-MDMA cohorts exhibited an oxytocin response compared with the placebo group.

### 5-Dimensions-Altered States of Consciousness (5D-ASC)

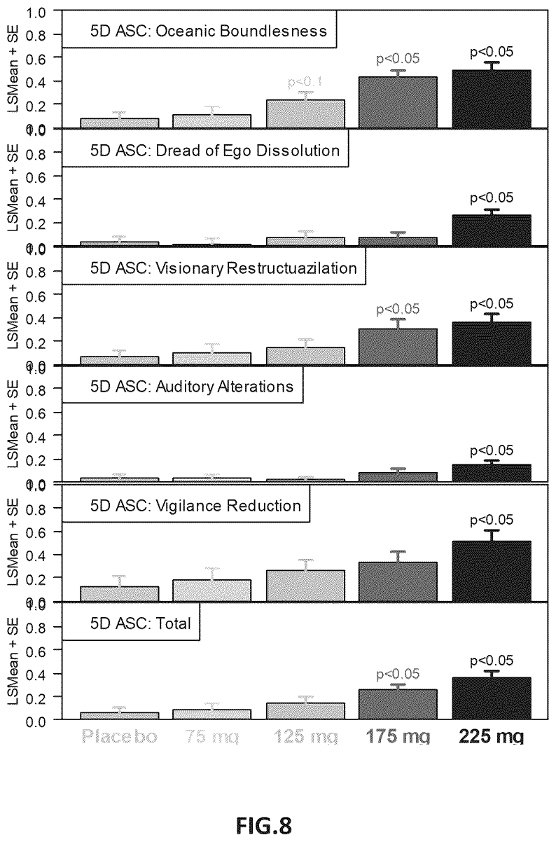

Consciousness questionnaire (5D-ASC) is utilized to evaluate the safety and/or efficacy of R(−)-MDMA. The 5D-ASC measures the acute drug effects using 5 primary dimensions and 11 lower-order scales to assess alterations in mood, perception and experience of self in relation to environment and thought disorder. The 5 dimensions include oceanic boundlessness, anxious ego dissolution, visionary restructuralization, auditory alterations, and reduction of vigilance. The results of the 5D-ASC assessment suggest that R(−)-MDMA possesses a profile that is different from the profile observed for racemic MDMA, and presents more psychedelic-like characteristics. This is unexpected given the prior assessments and results reported for racemic MDMA, particularly as regards visual phenomena (Visual restructrualization). As shown in FIG. 8, subjects reported a dose-dependent increase in all measured sub-components and in the total score (e.g., Oceanic Boundlessness, Dread of Ego Dissolution, Visionary Restructuralization, Auditory Alterations, and Vigilance Reduction); all of which were robust, statistically significant, and dose dependent.

A notable result illustrated by the ASC data was the reporting ratio of feelings of Oceanic Boundlessness (OB) relative to Dread of Ego Dissolution (DED). The ratio can be a useful metric as they are inversely correlated with psychedelic-mediated antidepressant efficacy (OB positively correlated, DED negatively correlated). A very favorable ratio was observed over the dosage range. The OB-DED ratio was consistent with the 11-Dimensional Altered States of Consciousness (11D-ASC).

### 11-Dimensions-Altered States of Consciousness (11D-ASC)

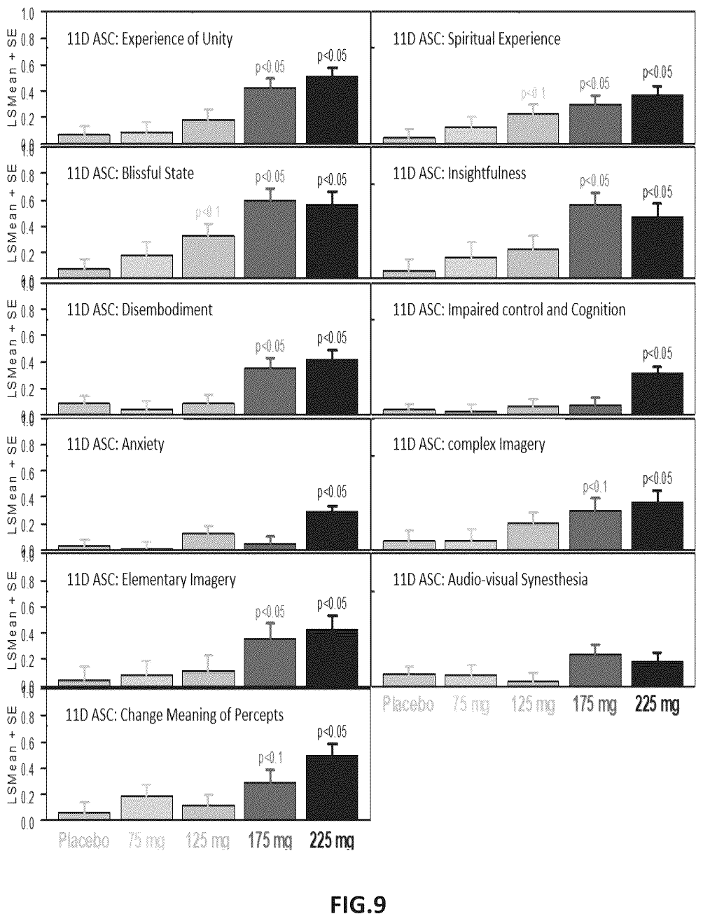

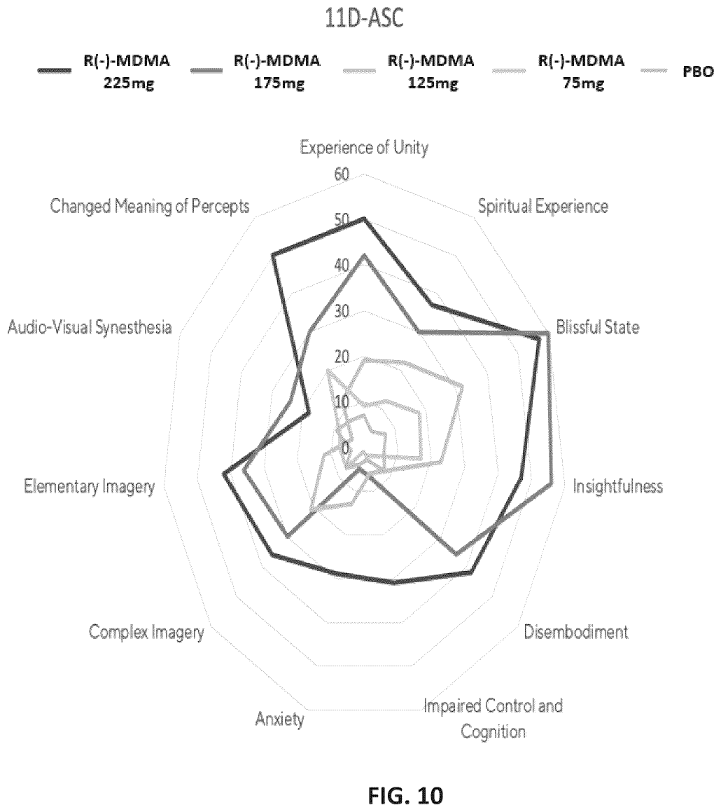

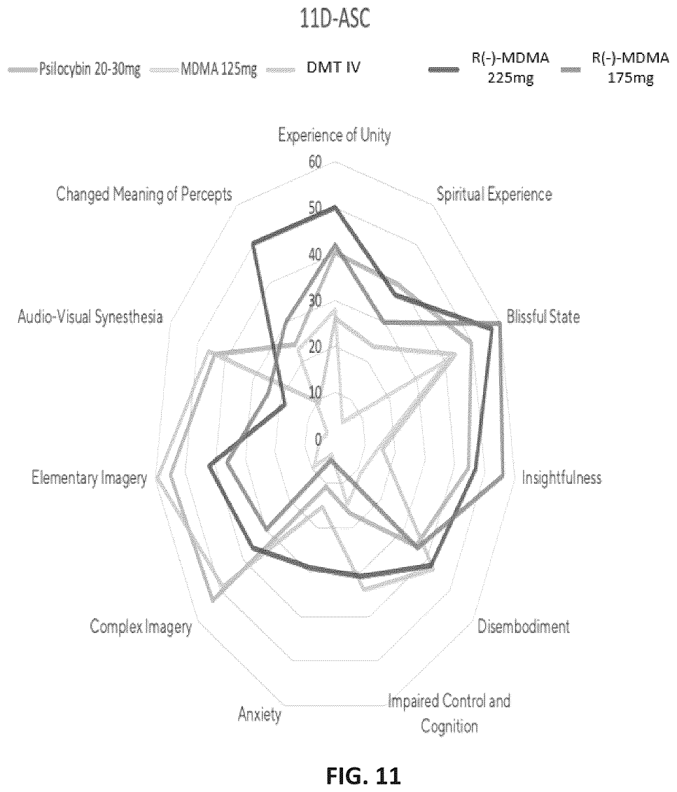

The results of the 11D-ASC assessment also show the unexpected psychedelic-like response associated with R(−)-MDMA. The 11-D results illustrate similar psychedelic-like response effects in both simple and complex imagery, perceptual alterations, and synesthesia (FIG. 9 and FIG. 10). In particular, FIG. 11 shows the comparison among psilocybin, intravenous dimethyltryptamine (IV DMT), racemic MDMA and R(−)-MDMA at 175 mg and 225 mg doses. The response profile observed for R(−)-MDMA has many similarities to psilocybin, with less imagery and more insights. Strikingly, the results for racemic MDMA are very different, with no visual hallucinations and little to no insight. Lastly, the effect of changed meaning of percepts, was strongest in the R(−)-MDMA 225 mg dose group. These features suggest that R(−)-MDMA can enable therapy that involves higher order cognition and reasoning, similar to what is often experienced with psilocybin, but with less risk of forming emotional attachment with the facilitator(s).

### Emotional Breakthrough Inventory (EBI)

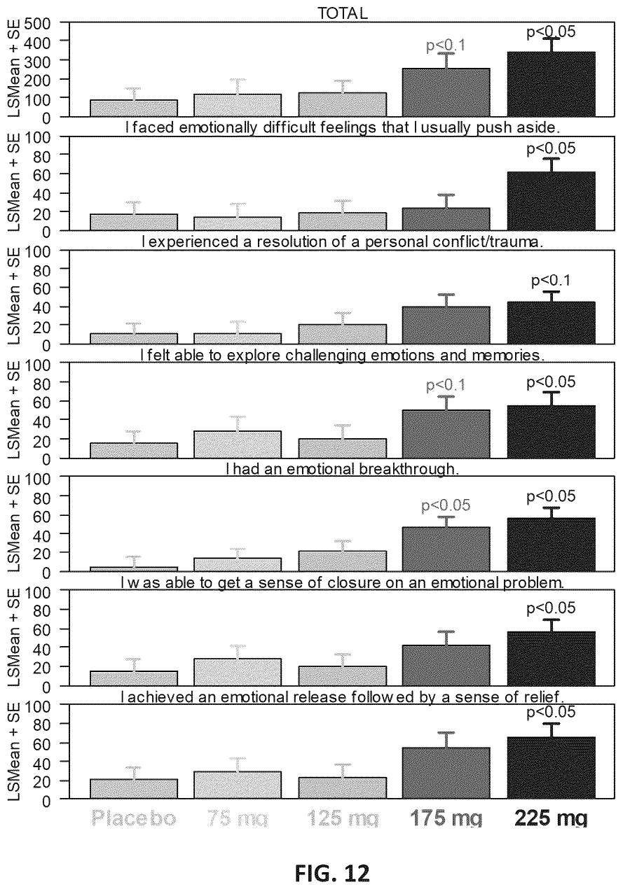

Self-reported Emotional Breakthrough Inventory (EBI) include facing emotionally difficult feelings that are usually pushed aside; experiencing resolution of a personal conflict or trauma; ability to explore challenging emotions and memories; having an emotional breakthrough; achieving a sense of closure on an emotional problem; and achieving emotional release followed by sense of relief. Emotional breakthrough is an important and distinct component of the acute psychedelic experience that appears to be a key mediator (state predictor) of subsequent longer-term psychological changes in psychiatric populations. (Carhartt-Harris, et al., Trial of Psilocybin versus Escitalopram for Depression, N Engl J Med 2021; 384:1402-1411). As illustrated in FIG. 12, R(−)-MDMA induced a dose-dependent increase in the acute experience of emotional breakthroughs that was significantly higher at 225 mg of R(−)-MDMA vs placebo. The effects of 225 mg of R(−)-MDMA on these acute experiences are similar to the magnitude of increases with the classic psychedelic, psilocybin.

### Facial Emotion Recognition Test (FERT)

R(−)-MDMA resulted in a numerical reduction in the recognition of angry (threatening) faces at all dose levels, (75 mg: 13.2%; 125 mg: 12.3%; 175 mg: 8.8%; 225 mg: 12.4%, and placebo: 6.6%) in the FERT. This reduction was similar to reductions reported in published reports of racemic MDMA which has been reported to reduce bias to angry faces by 12.5% (Hysek et al., 2014).

The core feature of SAD is the fear of being negatively evaluated by others and is manifested through the biased processing of facial expressions of negative emotions with hypervigilance/avoidance. The finding with 225 mg of R(−)-MDMA suggests a reduction in this core SAD pathological process of strongly perceiving threatening (e.g. angry) faces and also points to a potential attenuation of underlying neurobiological reactivity of these emotions.

Additionally, compared to placebo, 225 mg R(−)-MDMA also increased misclassifications to happy faces, which was when a face was labelled by a participant as “happy” when it was not a “happy” valenced face (Change from baseline: 225 mg: M: +1.16 (SD: 1.16); placebo: M: +0.95 (SD: 0.95). Attention bias to happy faces is believed to be part of the “social reward” motivational system activity.

### Multifaceted Empathy Test (MET)

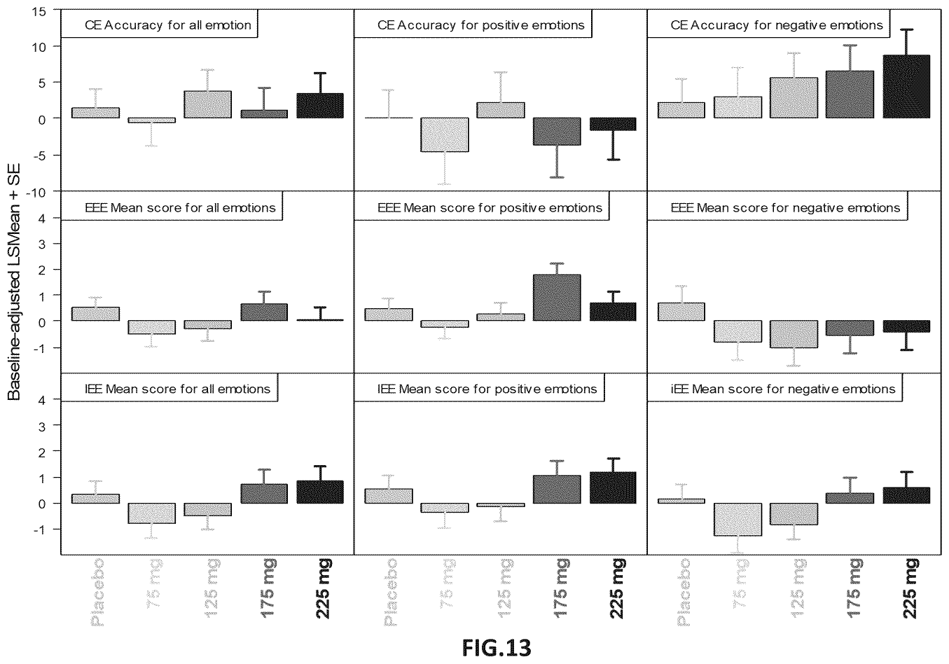

The Multifaceted Empathy Test (MET) measures cognitive empathy (CE), explicit emotional empathy (EEE) and implicit emotional empathy (IEE). (Hurlemann R, et al., (2010) Oxytocin enhances amygdala-dependent, socially reinforced learning and emotional empathy in humans. J Neurosci 30: 4999-5007.) The MET LSmean scores in CE negative emotions showed changes from baseline in a dose-dependent manner (FIG. 13). The results indicate that R(−)-MDMA can have positive effects on emotional and cognitive empathy. An increase in cognitive empathy accuracy suggests an improved ability to understand and interpret other people's emotions. An increase in implicit and explicit emotional empathy ratings suggests an improved ability to feel and share the emotions of others.

### Self-Compassion Scale Results

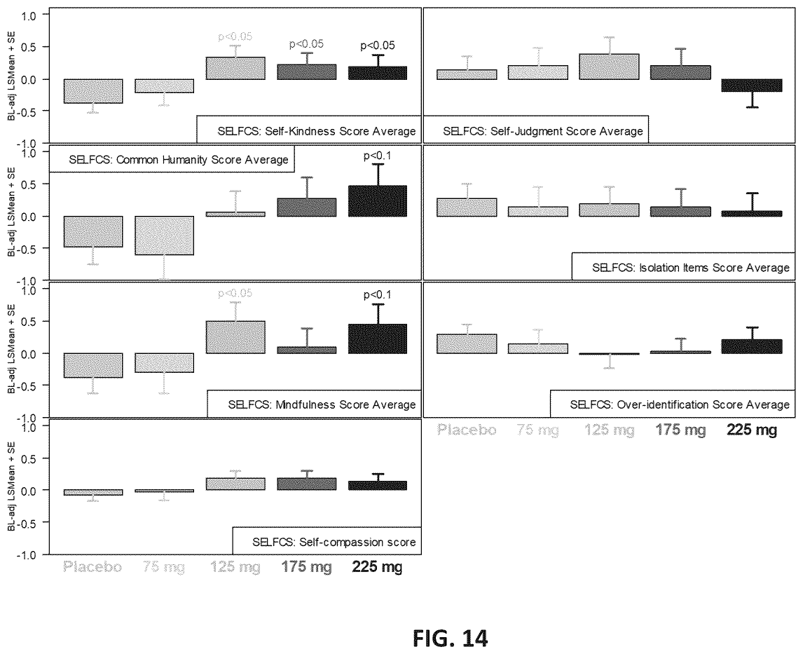

Self-compassion is defined as being open to and moved by one's own suffering, experiencing feelings of caring and kindness toward oneself, taking an understanding, non-judgmental attitude toward one's inadequacies and failures, and recognizing that one's own experience is part of the common human experience (Neff, K. D., 2003. The development and validation of a scale to measure self-compassion. Self and Identity, 2, 223-250). The Self-Compassion Scale is an assessment tool designed to measure three core components of self-compassion: self-kindness, common humanity and mindfulness. (SCS; Neff, K. D., 2011. Self-compassion: Stop beating yourself up and leave insecurity behind. New York, NY: Harper Collins) The total self-compassion score is the total of the scores of the sub-items: self-kindness, self-judgement (reversed), common humanity, isolation (reversed), mindfulness and over-identification (reversed). FIG. 14 shows the changes from baseline as measured by SCS on Day 8 following R(−)-MDMA dosing. Higher doses of R(−)-MDMA resulted in significantly increased self-compassion (self-kindness) scores at 1 week of follow-up. Self-kindness scores increased from a mean of 3.9 (SD=0.70) to 4.1 (SD=0.87) at 1 week of follow-up for participants receiving 225 mg R(−)-MDMA but decreased from a mean of 3.5 (SD=0.68) to 3.2 (SD=0.88) for participants receiving placebo.

### Challenging Experiences Questionnaire (CEQ)

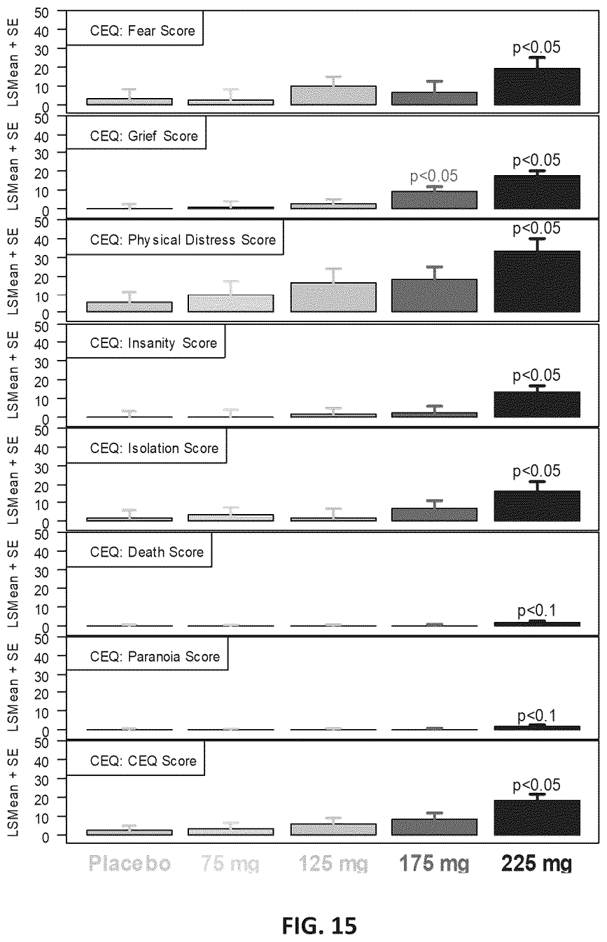

Seven Challenging Experiences Questionnaire (CEQ) factors (grief, fear, death, insanity, isolation, physical distress, and paranoia) provide a phenomenological profile of challenging aspects of experiences with R(−)-MDMA. As illustrated in FIG. 15, the changes from baseline at 6.5 hours post dosing showed a clear dose-dependent response with statistical significance at higher doses. These results also confirm the unexpected psychedelic-like response associated with R(−)-MDMA.

### Example 3. Use of R(−)-MDMA to Treat SAD

A Phase 2, randomized, double-blind, placebo-controlled trial will be conducted to evaluate the safety and efficacy of R(−)-MDMA in adult participants with Social Anxiety Disorder (SAD).

The primary objective is to assess the safety and tolerability of R(−)-MDMA in participants with SAD compared with Placebo (PBO). The endpoints include incidence and severity of adverse events (AEs), adverse events of special interest (AESIs) and serious adverse events (SAEs), incidence of clinically significant post-dosing abnormalities from physical examinations, vital signs, electrocardiogram (ECG) parameters, clinical laboratory results, and Columbia Suicide Severity Rating Scale score (C-SSRS).

The secondary objective is to evaluate the potential therapeutic efficacy by examining the effect size of R(−)-MDMA compared with PBO in improving social anxiety symptoms in participants with SAD. The endpoint includes change in Leibovitz Social Anxiety Scale (LSAS) total score.

The exploratory objectives include: (1) to assess the acute subjective effects of a single dose of R(−)-MDMA compared with PBO in participants with SAD; (2) to assess the therapeutic potential of R(−)-MDMA compared to PBO in improving participant and physician reported outcomes of anxiety, depressive, and quality of life in participants with SAD; and (3) to assess the pharmacokinetics (PK) of R(−)-MDMA and its metabolites following a single-dose of R(−)-MDMA. The corresponding endpoint includes: (1) mean sub-scale scores on the 5 Dimensions-Altered States of Consciousness (5D-ASC) at each assessment timepoint, mean sub-scale scores on the Challenging Experience Questionnaire (CEQ) at each assessment timepoint, and mean Subjective Intensity Rating Scale (SIRS); (2) change from baseline in mean scores in Clinician-rated Clinical Global Impression (CGI-I) Improvement score, Self-reported Social Phobia Inventory (SPIN) total score, Quick Inventory of Depressive Symptomatology (QIDS)-16 Items (QIDS-SR-16) total score, LSAS total score, LSAS sub-scale scores, Subtle Avoidance Frequency Examination (SAFE) total scores, and proportion of treatment responders (defined as ≤50 LSAS Total Score from Baseline); and (3) plasma PK parameters of R(−)-MDMA (including Tₘₐₓ, Cₘₐₓ, AUC₀₋ₗₐₛₜ and AUC₀₋₂₄), urine PK parameters of R(−)-MDMA, and metabolite profiling for presence of circulating metabolites in plasma.

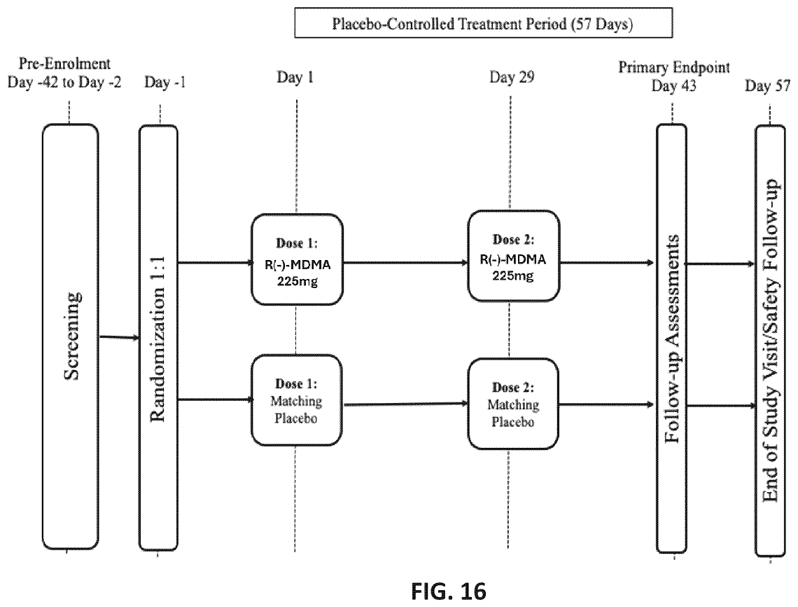

The phase 2 trial will enroll patients with SAD and groups will be randomized 1:1 to receive 2 oral doses of 225 mg R(−)-MDMA or placebo during the blinded treatment period. The Dosing Periods will be separated by 28 days. The primary endpoint will be assessed two weeks after receiving the second blinded R(−)-MDMA or placebo dose (Day 43). Participants will be monitored for safety and to estimate the trajectory of their symptoms until the End of Study (EOS) Visit (Day 57). The study design for phase 2 trial is provided in FIG. 16.

### Inclusion Criteria

Participants are eligible to be included in the study only if all the following criteria apply:

Participants must be aged between 18 and 65 years of age inclusive at the time of signing the informed consent.

Participants have current diagnosis of SAD, as defined by the Diagnostic and Statistical Manual of Mental Disorders, Fifth Edition Text Revision (DSM-5-TR), which is not better attributable to another psychiatric condition or to a medical condition. The diagnosis will be confirmed by the Mini-International Neuropsychiatric Interview (MINI). Because SAD may be underdiagnosed, medical records that indicate that SAD is likely but not formally diagnosed, or misdiagnosed as generalized anxiety disorder (GAD) for example, may be acceptable. In such cases, consult with the treating the medical monitor and Sponsor physician may be necessary to help solidify diagnosis.

Participants have clinician-administered Liebowitz Social Anxiety Scale total score ≥70 at screening.

Participants have CGI-Severity score ≥4 at screening.

Participants are willing to discontinue the use of anti-anxiety medications at least 4 weeks prior to Dosing Visit (Day 1) until after the completion of end of study (EOS); are willing to discontinue the use of antidepressant medications at least 4 weeks prior to the first dose of IMP (Day 1) until after the completion of the EOS (Day 57) Visit.

Existing medication regimens for other medical conditions should be stable for 6 weeks prior to Baseline, with the intent to remain stable until BOS,

### Exclusion Criteria

Participants who meet any of the following criteria will be excluded from this study:

Participants have a current or prior DSM-5-TR diagnosis of a schizophrenia spectrum and other psychotic disorder, substance/medication-induced psychotic disorder, bipolar and related disorder, or any disorder with psychotic features (including MDD with psychotic features), as assessed by medical history and a structured clinical interview (version 7.0.2 MINI).

Participants have a current or prior DSM-5-TR diagnosis of a neurocognitive disorder, intellectual disorder, dissociative disorder, disruptive/impulse-control/conduct disorder, autism spectrum disorder, or cluster A and B personality disorder, as assessed by medical history and a structured clinical interview (MINI). Inclusion of individuals with a diagnosis of autism spectrum disorder level 1 may be considered at the discretion of the investigator if the participant no longer meets criteria for the condition and current functioning and/or subthreshold symptoms will not interfere with treatment or compliance in the study.

Participants have a current DMS-5-TR diagnosis of SAD performance only sub-type, posttraumatic stress disorder, acute stress disorder, obsessive-compulsive disorder, anorexia nervosa, bulimia nervosa, or any other comorbid psychiatric condition that dominates the clinical presentation and would interfere with experimental treatment.

Participants have current DMS-5-TR disorder, other than SAD, which is the primary focus of treatment. Note that participants with concurrent GAD are eligible for the study, provided that GAD is not the primary diagnosis. Participants with attention deficit hyperactivity disorder (ADHD) are eligible for the study, provided that they do not require pharmacological treatment for the condition AND if ADHD is not the primary diagnosis.

Participants have severe current depression as measured by the Quick Inventory of Depressive Symptomatology (QIDS-SR-16) (Total Score ≥16).

Participants have a history of moderate or severe alcohol or cannabis use disorder within 1 year before Screening. Has any severity (including mild) of other substance use disorder (drug) within 1 year before Screening. The diagnosis of a substance use disorder and any associated severity grading, which is determined by standardized clinical specifiers, will be assessed by the MINI diagnostic interview at Screening.

Participants have had suicidal ideation with some intent to act within 6 months before Screening or a history of suicidal behavior within the past 1 year before Screening. Suicide risk should be informed by clinical judgment, excluding participants who score ≥17 on the MINI Suicidal Scale at Screening, or endorse “Yes” on Item 4 (active suicidal ideation with some intent to act, without specific plan) or Item 5 (active suicidal ideation with specific plan and intent) on the C-SSRS at Screening or Day −1.

Participants have a history of, or current signs and symptoms of, diseases or conditions that would mean that participation was not in the participant's best interest (e.g., compromise their wellbeing), or that could prevent, limit, or confound the protocol-specified assessments.

Participants have a history of moderate or severe head trauma (e.g., loss of consciousness for more than 15 minutes) or other neurological disorders (including a diagnosis of epilepsy or a seizure in the last 6 months), neurodegenerative disorder (Alzheimer's disease, Parkinson's disease, multiple sclerosis, Huntington's disease, etc.) or systemic medical diseases that are, in the opinion of the investigator, likely to interfere with the conduct of the study or confound the study assessments. A history of febrile seizures in childhood is not exclusionary.

Participants have a history of clinically significant cardiovascular, cerebrovascular, or peripheral vascular disease or condition including, but not limited to, Wolff Parkinson White syndrome, unstable angina, myocardial infarction, congestive heart failure, cardiac arrhythmia, valvular heart disease, hypertension, hypotension, bradycardia, or tachycardia.

Participants have elevated blood pressure at Screening, Day −1 or Day 1 (pre-dose) (SBP >140 mm Hg or DBP >90 mm Hg) or any past history of hypertension or hypertensive crisis. An abnormal blood pressure (BP) measurement at Screening may be repeated once, after the participant has relaxed for 10 to 15 minutes, to determine eligibility.

Participants have an active malignancy, or history of malignancy, excluding basal or squamous cell carcinoma of the skin or carcinoma in situ of the cervix, within 2 years prior to Screening.

Participants have any known allergy or hypersensitivity to R(−)-MDMA, racemic MDMA, any of the excipients in the formulation, or any history of prior exposure to R(−)-MDMA.

Participants have experienced symptoms of acute illness, infectious condition (e.g., coronavirus disease-2019) or chronic disease within 14 days before Screening or at Day −1, or any disease or condition (medical or surgical) that, by in the investigator's opinion, might compromise interpretation of safety or PK data, or would place the participant at risk because of their participation in the study.

Participants have received any prohibited therapies a summarized below:
a. Receipt of a known strong inhibitor or inducer of hepatic cytochrome P450 (CYP) 2D6 within 1 week or within a period 5 times the drug's half-life, whichever is longer, before Screening or Baseline.
b. Treatment with medications that are predominantly or exclusively substrates of CYP2D6 and with a narrow therapeutic index (sensitive CYP2D6 substrates) within 1 week or 5 times the drug's half-life, whichever is longer, prior to Screening.
c. Treatment with any antipsychotic medication or mood stabilizers within 4 weeks (6 months for long-acting injectables) prior to Screening.
d. Treatment with fluoxetine within 1 week prior to Screening or monoamine oxidase inhibitors (MAOIs) within 4 weeks prior to Screening.
e. Immunization with live attenuated vaccine within 4 weeks prior to Screening.
f. Chronic (i.e. daily) treatment with any anxiolytic medication, including benzodiazepines, within 4 weeks prior to Screening,
g. Use of any herbal remedies, which are psychoactive (e.g., St. John's Wort, kava kava, tryptophan, 5-hydroxytryptophan (5-HTP), valerian) within 1 week prior to Screening.
h. Any changes in psychotropic medication type or dose, with the exception of antidepressant taper, within 6 weeks prior to Screening.
i. During the antidepressant medication taper during screening the participant is deemed at-risk for ‘discontinuation syndrome’ as defined by a total score of ≥4 on the Discontinuation Emergent Sign and Symptoms (DESS) checklist.

Participants have recently initiated psychotherapy (e.g., CBT, Group Therapy, Interpersonal Psychotherapy, Psychodynamic Psychotherapy other than psycho-education) within 90 days prior to Screening. Participants can be concurrently receiving psychotherapy during the trial, as long as they have been engaged in stable psychotherapy treatment for at least 90 days prior to Screening and are planning to remain in treatment until completion of the EOS (Day 57) Visit. Participants who are currently not receiving any form of counselling or psychotherapy must be willing to avoid starting any of these treatments until the completion of the EOS (Day 57) Visit.

Participants have four or more documented failed treatment trial with a registered medication approved for SAD, at any time during the lifetime of the participant, whereby an adequate treatment trial is defined as that described in the package insert for a particular drug during which the participant received an adequate medication dosage (defined as the treatment dose indicated in the package insert to obtain efficacy for that particular drug). Failure is defined as less than 50% improvement or based on clinical judgment.

Participants have received electroconvulsive therapy, transcranial magnetic stimulation, vagal nerve stimulation, deep brain stimulation, or other brain stimulation treatment within 6 months prior to Screening, or is currently receiving these procedures.

Participants have received treatment with another investigational drug, investigational device, or approved therapy for investigational use within 90 days or 5 half-lives (whichever is longer) prior to Screening; prior participation at any time in non-invasive methodology trials in which no drugs were given is acceptable.

Participants have a positive toxicology panel (urine test, including qualitative identification of amphetamines, barbiturates, cocaine, ecstasy (3,4 methylenedioxymethamphetamine [MDMA]), methadone, methamphetamines, and opiates), or a positive alcohol breath test result at Screening or pre-dose on Day 1. A participant with a positive urine drug test result at Screening due to prescribed opiates may be permitted to continue screening if the prohibited medication is discontinued at least 1 week or 5 half-lives (whichever is longer) prior to the first dose of R(−)-MDMA. A positive toxicology screen for cannabinoids is permissible at Screening, if the participant does not meet criteria for moderate or severe cannabis use disorder, since mild cannabis use, which has been found to be disproportionately more common in the SAD population, but unlikely to interfere with the known pharmacological effects of the mechanism of action of R(−)-MDMA. Retesting is not permitted for positive urine drug test result(s) from any other nonprescription use of drugs of abuse.

Clinically significant vital sign measurements at Screening, Day −1, or Day 1 (pre-dose). Clinically significant Screening values measured after 5 minutes of rest in a supine or semi-supine position include:
a. Abnormal systolic blood pressure (SBP) (<90 or >140 mm Hg);
b. Abnormal diastolic blood pressure (DBP) (<40 or >90 mm Hg); or
c. Abnormal respiration rate (RR) (<10 or >22 breaths per minute),
d. Abnormal heart rate (HR) (<55 bpm or >100 bpm)

Vital signs measurements may be repeated once, at the investigator's discretion.

Participants have clinically significant laboratory abnormalities at Screening including any of the following:
a. Hemoglobin ≤130 g/L (for participants assigned male at birth) or ≤115 g/L (for participants assigned female at birth); or
b. Alanine aminotransaminase (ALT) or aspartate aminotransaminase (AST) values ≥2× upper limit of normal (ULN) or total bilirubin >2×ULN. Note: Abnormal laboratory values can be retested once to confirm results prior to screen failing a participant, and participants with Gilbert's syndrome are not excluded.
c. Potassium <3.5 mEq/L (3.5 mmol/L), indicative of mild or severe hypokalemia.

Participants have a clinically significant history or presence of abnormal ECG findings, as judged by the investigator, at Screening or Day 1 (pre-dose), including:
a. QT interval corrected according to Fridericia's formula (QTcF) interval >450 msec (for participants assigned male at birth) or >470 msec (for participants assigned female at birth);
b. History of risk factors for QT interval prolongation;
c. Evidence of 2nd and 3rd degree atrioventricular block, complete left bundle branch block, or complete right bundle branch block;
d. Features of new ischaemia; or
e. Arrhythmia (except premature atrial contractions and premature ventricular contractions).

Participants have positive test results for hepatitis B surface antigen (HBsAg), hepatitis C virus (HCV) antibody (anti-HCV), or anti-human immunodeficiency virus (HIV) type 1 antibody (anti-HIV) at Screening; participants with prior recovered hepatitis B virus infection will be included with confirmed normal liver function test results; and participants with positive anti-HCV results, but who have been effectively treated for hepatitis C as evidenced by a negative hepatitis C virus ribonucleic acid confirmation test, and who no longer require antiviral therapy, are eligible for participation.

Participants who are lactating or pregnant as confirmed by a serum pregnancy test at Screening and negative urine pregnancy test before dosing (applies only to participants of childbearing potential [POCBP]).

If known, participants have a biological first degree relative with schizophrenia, bipolar I/II, and/or psychotic disorder (unless induced by substance or medical condition).

Participants reports any lifetime use of MDMA or MDMA-containing drugs or reports a history of >2/lifetime administrations of psychedelic drugs, and/or last use of a psychedelic drug within 6 months prior to Screening or Day −1, or has reported hallucination persisting perception disorder (HPPD) or other significant AEs after prior use of hallucinogens.

Participants have experienced symptoms of acute illness or chronic disease within 14 days prior to Screening or Day −1, or any disease or condition (medical or surgical) that, by the determination of the PI, might compromise interpretation of the study data, or would place the participant at risk as a result of participation in the study.

Participants are unable to cooperate fully with the requirements of the study protocol, including the SoA, or is likely to be non-compliant with any study requirements.

Other unspecified reasons that, in the opinion of the PI or Sponsor, make the participant unsuitable for enrollment.

Participants have poor venous access.

Participants have donated blood or plasma within 30 days prior to Screening, lost more than 500 mL of whole blood within the 30 days prior to Screening, or received a blood transfusion within 1 year prior to Screening.

### Psychological Support

All participants will be provided with standardized psychological support prior to, during, and post-administration of R(−)-MDMA from a facilitator throughout the trial. The participant will remain with the same facilitator throughout the duration of the trial. Psychological support is specific to the R(−)-MDMA experience and does not constitute psychotherapy or follow a specific therapeutic model.

1. A method of treating an anxiety disorder in a subject in need thereof, comprising administering to the subject a composition comprising an enantiomerically enriched form of R(−)-3,4-methylenedioxymethamphetamine (R(−)-MDMA), or a pharmaceutically acceptable salt or prodrug thereof,
wherein the administration provides a R(−)-MDMA Tₘₐₓ of about 2 hours to about 10 hours and a R(−)-MDMA Cₘₐₓ of about 150 ng/ml to about 3000 ng/ml following administration.

2. The method of claim 1, wherein the anxiety disorder is social anxiety disorder, or generalized anxiety disorder.

3. (canceled)

4. The method of claim 1, wherein the subject has one or more conditions comorbid with the anxiety disorder.

5. The method of claim 4, wherein the one or more conditions comorbid with an anxiety disorder is a mood disorder, a depressive disorder, an anxious distress, a rejection sensitivity, a mixed anxiety and depressive disorder (MADD), a major depressive disorder, or a treatment resistant depression, a bipolar disorder, schizophrenia, an eating disorder, an attention deficit/hyperactivity disorder, epilepsy, a cardiovascular disease, a substance abuse disorder, migraine, a headache disorder, an irritable bowel syndrome, a dementia, post-traumatic stress disorder (PTSD), Alzheimer's disease, Parkinson's disease or any combination thereof.

6. The method of claim 5, wherein the eating disorder comprises anorexia nervosa, bulimia nervosa, or binge eating, or
wherein the substance abuse disorder comprises alcohol abuse, caffeine abuse, cannabis abuse, hallucinogen abuse, inhalant abuse, opioid abuse, sedative abuse, hypnotic abuse, anxiolytic abuse, stimulant abuse, or tobacco abuse.

7. (canceled)

8. The method of claim 6, wherein the hallucinogen is selected from phencyclidine or similarly acting arylcyclohexylamines, and lysergic acid diethylamide (LSD), or wherein the stimulant is selected from amphetamine-type substances and cocaine.

9. (canceled)

10. The method of claim 1, wherein about 75 mg to about 350 mg, about 75 mg to about 225 mg, about 75 mg, about 125 mg, about 175 mg, or about 225 mg R(−)-MDMA free base or an equivalent dose of the pharmaceutically acceptable salt or prodrug thereof is administered to the subject.

11. (canceled)

12. (canceled)

13. (canceled)

14. The method of claim 1, wherein the subject is administered 2 repeated doses of the composition at an interval of about 14 days to about 56 days,
wherein the subject is administered 2 repeated doses of the composition at an interval of about 28 days, or
wherein the subject is administered once about every about 2 weeks to about 6 months.

15. (canceled)

16. (canceled)

17. The method of claim 1, wherein administering the composition comprises injection, oral delivery, transdermal delivery, or transmucosal delivery.

18. (canceled)

19. The method of claim 1, further comprising administering a second therapy.

20. The method of claim 19, wherein the second therapy comprises a psychological support.

21. The method of claim 1, wherein the composition comprises a weight ratio of R(−)-MDMA to S(+)-MDMA ranging from about 50.1:49.9 to about 100:0.

22. The method of claim 1, wherein the composition comprises R(−)-MDMA in an enantiomerically enriched form of ≥90%, ≥95%, ≥ 96%, ≥97%, ≥98%, ≥99%, ≥99.5%, ≥99.9%, or ≥99.99%, or 100% relative to total MDMA.

23. The method of claim 1, wherein the composition comprises R(−)-MDMA in an enantiomeric excess to S(+)-MDMA from about 95% to about 100%, or about 98% to about 100%.

24. (canceled)

25. The method of claim 1, wherein the administration provides a R(−)-MDMA Tₘₐₓ of about 3 hours to about 8 hours, or about 3 hour to about 5 hours.

26. (canceled)

27. The method of claim 1, wherein the administration provides a R(−)-MDMA AUC₀₋ₜ of about 2000 ng·h/mL to about 20,000 ng·h/mL following administration.

28. The method of claim 1, wherein the subject has SAD that is not a performance only sub-type.

29. The method of claim 1, wherein the subject has a Liebowitz Social Anxiety Scale (LSAS) total score ≥70 prior to the administration, wherein the subject has Clinician Global Impressions-Severity (CGI-S) score ≥4 prior to the administration, or a combination thereof.

30. The method of claim 29, wherein the administration provides a reduction in the subject's LSAS total score by at least about 20 compared to prior to the administration, wherein the subject's LSAS total score is reduced to no greater than 50 after the administration, wherein the administration provides a reduction in the subject's LSAS sub-scale score by at least about 10% compared to prior to the administration, or any combination thereof.

31. (canceled)

32. (canceled)

33. (canceled)

34. The method of claim 1, wherein the administration provides:
(1) a reduction in the subject's Clinician-rated Clinical Global Impressions-Improvement (CGI-I) score by at least about 10% compared to prior to the administration;
(2) the administration provides a reduction in the subject's total and sub-scale scores on the 5D-ASC rating scale by at least about 10% compared to prior to the administration;
(3) a reduction in the subject's sub-scale scores on the Challenging Experience Questionnaire (CEQ) by at least about 10% compared to prior to the administration;
(4) an increase in the subject's Spoken Intensity Rating Scale (SIRS) by at least about 10% compared to prior to the administration;
(5) an increase in the subject's Self-reported Patient Global Impressions of Change (PGI-C) score by at least about 10% compared to prior to the administration;
(6) a reduction in the subject's self-reported Social Phobia Inventory (SPIN) total score and sub-scale scores by at least about 10% compared to prior to the administration;
(7) a reduction in the subject's Quick Inventory of Depressive Symptomatology (Self Report) 16 item (QIDS-SR-16) total score by at least about 10% compared to prior to the administration;
(8) a reduction in the subject's Subtle Avoidance Frequency Examination (SAFE) total score by at least about 10% compared to prior to the administration; or
any combination thereof.

35. (canceled)

36. (canceled)

37. (canceled)

38. (canceled)

39. (canceled)

40. (canceled)

41. (canceled)
